(19) United States
(12) Patent Application Publication
(10) Pub. No.: US 2012/0329738 A1
(43) Pub. Date: Dec. 27, 2012

(54) Water Soluble Drug-Solubilizer Powders and Their Uses

(76) Inventor: Zhijun Liu, Baton Rouge, LA (US)

(21) Appl. No.: 13/501,496

(22) PCT Filed: Oct. 15, 2010
(86) PCT No.: PCT/US10/52786

§ 371 Date: Jun. 14, 2012

Related U.S. Application Data
(60) Provisional application No. 61/251,768, filed on Oct. 15, 2009.
(60) Provisional application No. 61/314,800, filed on Mar. 17, 2010.

(51) Int. Cl. A61K 47/26; A61K 31/337; A61K 31/7048; A61K 31/704

(52) U.S. Cl. 514/26; 514/777; 514/449; 514/27

(57) ABSTRACT

Enhanced methods have been discovered, using either sonication or homogenization followed by increased temperature and pressure, to solubilize compounds using diterpene glycosides and to produce a powder form of the compound-solubilizer complex than can be reconstituted in water. Without the diterpene glycoside, the compounds were insoluble or sparingly soluble in water, including some fat-insoluble vitamins. Water solutions of these compounds were made using a diterpene glycoside solubilizer, for example, rubusoside. The compound-solubilizer complex was then dehydrated to a stable powder that could then be reconstituted with water. A reconstituted drug-solubilizer complex (curcumin-rubusoside) was shown to be effective on reconstitution. In addition, the diterpene glycoside, rubusoside, was shown to be an inhibitor of permeability glycoprotein (P-gp), and will thus increase gastrointestinal absorption of certain drugs administered with rubusoside.

(In countries other than the United States:) The benefit of the 15 Oct. 2009 filing date of U.S. provisional patent application 61/251,768 and the benefit of the 17 Mar. 2010 filing date of Unites States provisional patent application 61/314,800 are claimed under applicable treaties and conventions. (In the United States:) The benefit of the 15 Oct. 2009 filing date of U.S. provisional patent application 61/251,768 and the benefit of the 17 Mar. 2010 filing date of Unites States provisional patent application 61/314,800 are claimed under 35 U.S.C. §119(e) in the United States.

### TECHNICAL FIELD

This invention pertains to a powder that contains drug-solubilizer complexes that can be reconstituted in water, including several fat soluble vitamins. In addition, a more efficient method to make the solubilizers-drug complex has been found. The drug-solubilizer complexes that contain rubusoside have also been found to inhibit permeability glycoprotein and thus improve gastrointestinal adsorption of the drug-solubilizer complex.

### BACKGROUND ART

### Important Compounds Insoluble in Water

Poor aqueous solubility is a common obstacle to delivering pharmaceuticals or other bioactive compounds and is a major challenge in formulating new drug products. In a study of kinetic aqueous solubility of commercial drugs, 87% were found to have solubility in water of ≧65 μg/mL and 7%≦20 μg/mL (Lipinski, C., et al., Adv. Drug Deliv. Rev. (1997) 23:3-25). The minimum acceptable aqueous solubility for a drug is about 52 μg/mL solubility based on 1 mg/kg clinical dose and average permeability (C. A. Lipinski, J Pharm Tox Meth (2000) 44:235-249). The pharmaceutical industry has been employing various approaches to increasing water-insoluble drugs for pharmaceutical drug formulations. Commonly used approaches are the uses of one or more complexing agents (e.g., cyclodextrins), cosolvents (e.g., ethanol, polyethylene glycol), surfactants (e.g., Cremophor EL, Tween 80), emulsifiers (e.g., lecithin, glycerol), and liposome, and nanosuspension techniques, alone or in combinations. Within this group, the use of complexing agents to improve solubility of water-insoluble drugs is increasing. Complexing agents improve water solubility by forming a non-covalent stoichiometric association with the pharmaceutical drug. Currently, the main complexing agents in the pharmaceutical industry are various forms of cyclodextrins (“CDs,” molecular weight around 1135 Daltons), which form inclusion complexes with water-insoluble drug. The use of cyclodextrin inclusion complexation has successfully solubilized many insoluble drugs, including an antifungal, voriconazole, and an antipsychotic, ziprasidone mesylate, which use sulfobutylether-β-cyclodextrin as the complexing agent. The most important cyclodextrins are parent α-, β-, and γ-CD as well as two modified hydroxypropyl-β-CD and sulfobutylether-β-CD. However, even the use of cyclodextrins has its disadvantages. Some of these limitations include lack of compatibility of the drug molecules with the inclusion cavity of CDs, precipitation of the formed complexes of CD-drug during dilution (e.g., in the stomach), potential toxicity and quality control of uniform CDs, and low complexation efficiency for achieving desirable solubility effect. Therefore, new complexing agents that are superior to cyclodextrins in overcoming or reducing these limitations are needed for the formulations of pharmaceutical, cosmetic, agricultural chemicals, and foods products.

Diterpenes.

Taxanes are diterpenes produced by the plants of the genus Taxus (yews) such as the Pacific Yew (Taxus brevifolia) in the family of Taxaceae. Taxanes include paclitaxel and docetaxel. Paclitaxel is the anti-cancer drug under the drug name of TAXOL® and docetaxel is used under the name of TAXOTERE® (Medicinal Natural Products—A Biosynthetic Approach, 1997, John Wiley & Sons, Chichester, England; pp 186-188). Paclitaxel is an anti-cancer diterpenoid alkaloid and is not soluble in water. The structure of paclitaxel is shown in FIG. 1H. Therapeutic solutions of paclitaxel currently contain either an oil or dehydrated alcohol or both; or paclitaxel is bound to albumin. None of these formulations are true water solutions. Other taxanes include baccatin III, 10-deacetylbaccatin III, cephalomannine, and 10-deacetylcephalomannine. These taxanes are characterized with a four-membered oxetane ring and a complex ester side-chain in their structures. All taxane compounds have poor water solubility. (U.S. Patent Application Publication no. 2007/0032438). Other medicinally important, but insoluble or poorly soluble diterpenes include retinoids (vitamin A, retinol (vitamin A1), dehydroretinol (vitamin A2), retinoic acid, β-cis-retinoic acid and other retinol derivatives, ginkgolides, and forsakolin (a promising drug for the treatment of glaucoma, congestive heart failure, and bronchial asthma).

Quinoline Alkaloids.

Quinoline alkaloids are alkaloids that possess quinoline in their structures and are terpenoid indole alkaloid modifications. Camptothecins isolated from the Camptotheca acuminata trees (Family Nyssaceae) are quinoline alkaloids. Camptothecin (CPT) is a cytotoxic alkaloid and is reported to have anti-tumor properties, perhaps by inhibiting topoisomerase 1. (See, for example, U.S. Pat. No. 4,943,579). The structure of camptothecin is shown in FIG. 1F. It has poor solubility in water (The Merck Index, 1996). Semi-synthetic analogues of camptothecins such as topotecan and irinotecan are approved chemotherapeutic drugs. Natural camptothecins include camptothecin, 10-hydroxycamptothecin, methoxycamptothecin, and 9-nitrocamptothecin. None of the natural camptothecins are water soluble (see, for example, US Patent Application Publication no. 2008/0242691). Camptothecins have broad-spectrum anti-cancer activity, but poor water solubility has limited direct uses as chemotherapeutic agents. Other quinoline alkaloids include the long recognized anti-malarial drugs quinine, quinidine, cinchonidine, and cinchonine.

Phenylalanine-Derived Alkaloids.

Phenylalanine-derived alkaloids are compounds that either possess or derive from phenylalanine ring structures, e.g., capsaicin and dihydrocapsaicin. Capsaicin (CAP) is a pungent phenylalanine alkaloid derived from chili peppers and can desensitize nerve receptors. The structure of capsaicin is shown in FIG. 1G. It is practically insoluble in cold water (The Merck Index, 1996).

Hydrolysable Tannins.

Hydrolysable tannins include gallotannins, which include gallic acid and compounds with gallic acid as the basic unit, and ellagitannins, which include ellagic acid and compounds with ellagic acid as the basic unit. The structure of gallic acid is shown in FIG. 1A. Gallic acid is reported to be both an antioxidant and antiangiogenic agent (See, for example, Published International Application WO 2005/000330). Gallic acid is sparingly soluble (about 11 mg/ml) in water at room temperature, and the solution is light sensitive (The Merck Index, 1996).

Flavonoids. Flavonoids are polyphenolic compounds, and include flavonoids derived from a 2-phenylchromen-4-one (2-phenyl-1,4-benzopyrone) structure, isoflavonoids derived from a 3-phenylchromen-4-one (3-phenyl-1,4-benzopyrone) structure, and neoflavonoids derived from a 4-phenylcoumarine (4-phenyl-1,2-benzopyrone) structure. Many chalcones act as precursors to form a vast variety of flavonoids. The most noticeable subclasses of flavonoids include flavonones (e.g., naringenin and eriodictyol), flavones (e.g., apigenin and luteolin), dihydroflavonols (e.g., dihydrokaempferol and dihydroquercetin), flavonols (e.g., kaempferol and quercetin), flavandiols and leucoanthocyanidins (e.g., leucopelargonidin and leucocyanidin), water-soluble catechins (e.g., afzalechin and catechin), moderately soluble anthocyanidins (e.g., pelargonidin and cyaniding), as well as flavonol glycosides (e.g., rutin) and flavonone glycosides (e.g., hesperidin, neohesperidin and naringin). Isoflavonoids include, for example, the compounds daidzein and genistein (phytooestrogens). Neoflavonoids include, for example, the compounds of coumestrol, rotenone, and pisatin. A specific example of a flavonol glycoside is rutin, a light-yellow colored compound, which is a potent anti-oxidant that inhibits some cancers and reduces the symptoms of haemophilia. The structure of rutin is shown in FIG. 1B. Rutin has also a veterinary use in the management of chylothorax in dogs and cats. The obstacle to all these potential uses is its poor solubility in water (125 μg/ml; The Merck Index, 1966).

Curcuminoids/Phenols.

Curcuminoids/phenols are a class of compounds found in turmeric spice from the plant, Curcuma longa, of the ginger family. Curcuminoids include, for example, curcumin, desmethoxycurcumin, and bis-desmethoxycurcumin. Other phenols include, for example, tocopherols (vitamin E), propofol, and gingerols. Curcumin is an orange-yellow pigment that is found in the rhizome of Curcuma longa, the source of the spice turmeric. The structure of curcumin is shown in FIG. 1E. Curcumin has been reported to have several beneficial properties, including promotion of general health, anti-inflammatory and antimicrobial properties, and treatment for digestive disorders. (See, for example, U.S. Pat. No. 6,673,843) Curcumin is a lipophilic compound that is insoluble in water (The Merck Index, 1996). Alpha-tocopherol, one of the most potent forms of Vitamin E, is a lipid-soluble phenol compound that is not soluble in water. Its structure is shown in FIG. 1N. Gingerols are lipid-soluble phenol compounds primarily isolated from the root of ginger (Zingiber officinale). The structure of 6-gingerol is shown in FIG. 1P. Gingerols (e.g., 6-gingerol) may reduce nausea caused by motion sickness or pregnancy and may also relieve migraine.

Propofol is a drug for anesthetic and hypnotic uses. Currently, there are two drug forms using propofol. Its structure is shown in FIG. 1O. Propofol is formulated as an emulsion of a soya oil/propofol mixture in water. Newer generic formulations contain sodium metabisulfite or benzyl alcohol. Propofol emulsion (also known as “milk of amnesia”) is a highly opaque white fluid. The drug is sold as 200 mg propofol in 20 mL emulsifier (1%). The other drug form of propofol is a water-soluble form of the drug, fospropofol.

Quinones.

Quinones are a class of compounds having a fully conjugated cyclic dione structure. This class includes, for example, ubiquinones (coenzyme Q, such as coenzyme Q10), plastoquinones, anthraquinones (e.g., rhein, emodin, alizarin, and lucidin), phenanthraquinones (e.g., cryptotanshinone, tanshinone I, tanshinone IIA, and dihydrotanshinone), and di-anthraquinones (e.g., sennosides A and B). For example, tanshinone HA is one of the natural analogues of tanshinone. The structure of tanshinone IIA is shown in FIG. 1C. Tanshinones have been reported to have various physiological activities from attenuating hypertrophy in cardiac myocytes to aiding in treatment of obesity. (See, for example, U.S. Patent Application Publication 2007/0248698). Tanshinone IIA (as well as other tanshinones such as tanshinone I) is soluble in methanol but insoluble in water.

Another quinone is coenzyme Q10 (often abbreviated as CoQ10), a benzoquinone. The structure of CoQ10 is shown in FIG. 1D. This oil-soluble vitamin-like substance is a component of an electron transport chain in aerobic cellular respiration. CoQ10 acts as an antioxidant and is often used as a dietary supplement. The problems with CoQ10 are its insolubility in water and low bioavailability. Several formulations have been developed and tested on animals or humans including attempts to reduce the particle size and increase surface area of the compound, soft-gel capsules with CoQ10 in oil suspension, the use of aqueous dispersion of solid CoQ10 with tyloxapol polymer, formulations based on various solubilising agents, i.e. hydrogenated lecithin, and complexation with cyclodextrins, carriers like liposomes, nanoparticles, and dendrimers. Solubilizing CoQ10 in a water solution could have many uses as new medical treatments, including the administration by injection.

Microlides.

Microlides are a large family of compounds, many with antibiotic activity, characterized by a macrocyclic lactone ring typically 12-, 14-, or 16-membered (reflecting the number of units used), but can also be even larger polyene macrolides with microlide ring size ranging from 26 to 38-membered. Some examples of typical macrolides are erythromycins (14-membered) from Streptomyces erythreus, oleandomycin (14-membered) from Streptomyces antibioticus, spiramycin I, II, and III (16-membered) from Streptomyces ambofaciens, tylosin (16-membered) from Streptomyces fradiae, and avermectins (16-membered with a long polyketide chain). Some examples of polyene macrolides are amphotericin B from Streptomyces nodosus, nystatin from Streptomyces noursei, tacrolimus (23-membered) from Streptomyces tsukubaensis, and rapamycin (sirolimus; 31-membered).

Erythromycin is a macrolide antibiotic (polyketide). Its structure is shown in FIG. 1J. Erythromycin has an antimicrobial spectrum similar to or slightly wider than that of penicillin, and is often used for people who have an allergy to penicillins. For respiratory tract infections, it has better coverage of atypical organisms, including mycoplasma and Legionella.

Amphotericin B is a polyene antifungal, antibiotic from Streptomyses and has antimicrobial spectrum covering yeast and other fungi. It is a yellowish powder that is insoluble in water. The structure of amphotericin B is shown in FIG. 1V. Examples of applications of Amphotericin B: (1) antifungal: use of intravenous infusion of liposomal or lipid complex preparations of Amphotericin B to treat fungal disease, e.g., thrush; (2) use in tissue culture to prevent fungi from contaminating cell cultures. It is usually sold in a concentrated lipid complex/liposomal solution, either on its own or in combination with the antibiotics penicillin and streptomycin; (3) use as an antiprotozoal drug in otherwise untreatable parasitic protozoan infections such as visceral leishmaniasis and primary amoebic meningoencephalitis; and (4) use as an antibiotic in febrile, immunocompromised patients who do not respond to broad-spectrum antibiotics. An aqueous formulation of amphotericin B would offer new ways to administer this important drug, including intravenous use.

Nystatin is polyene macrolide from Streptomyces noursei which increases the permeability of the cell membrane of sensitive fungi by binding to sterols. It has an antimicrobial spectrum against yeasts and molds. It is a light yellowish powder, and is relatively insoluble in water. The structure of nystatin is shown in FIG. 1K. Current administration orally or topically relies on formulations based on lipids. Examples of applications of nystatin include cutaneous, vaginal, mucosal and esophageal Candida infections; and as prophylaxis in patients who are at risk for fungal infections. A water soluble formulation will allow new uses and routes of administration.

Rapamycin, also known as Sirolimus, is an immunosuppressant drug used to prevent rejection in organ transplantation; it is especially useful in kidney transplants. The structure is shown in FIG. 1U. Rapamycin is a macrolide originally developed as an antifungal agent, but later as a potent immunosuppressive and antiproliferative drug. Recently, rapamycin has been the subject of research and development as an inhibitor of the mammalian target of rapamycin (mTOR) for the treatment of cancer (e.g., leukemia). Rapamycin is not soluble in water. An oral solution drug containing Sirolimus formulated in phosal 50 PG and Tween 80 is currently used to prevent rejection in organ transplantation. A water solution containing therapeutic amounts of rapamycin has not been available.

Cyclic Peptides.

Cyclic peptides are a class of antibiotic compounds composed of cyclic peptides produced mostly by fungi such as Cylindrocarpon lucidum and Tolypocladium inflatum. Examples of cyclic peptide compounds that are water insoluble are cyclosporine, polymyxins, tyrothricin, gramicidins, capreomycin, vancomycin, cephalosporins, and cephamycins. Cyclosporin A, also known as cyclosporine, is a fungal metabolite possessing potent immunosuppressive properties. It is a white powder that is insoluble in water. The structure of Cyclosporin A is shown in FIG. 1I. Cyclosporin A is administered orally and by injection in non-aqueous compositions, and current application relies upon suspensions and emulsions of the drug. Examples of applications of cyclosporin include an immunosuppressant drug in organ transplants to reduce the activity of the patient's immune system; use for several autoimmune disorders, inducing psoriasis, severe atopic dermatitis, and rheumatoid arthritis and related diseases; use as a neuroprotective agent in conditions such as traumatic brain injury; and use in several veterinary medicines, for example, keratoconjunctivitis sicca (“dry eye”) in dogs; perineal fistulas; atopic dermatitis in dogs; immune-mediated hemolytic anemia; discoid lupus erythemathosus (topical use); feline asthma; german shepherd pannus (ophthalmic preparation); and kidney transplantation.

Sesquiterpene Lactones.

Sesquiterpene lactones are a class of sesquiterpenes (15-carbon compounds) containing a lactone. Examples of insoluble sesquiterpenes are artemisinin (a new, highly-effective anti-malarial compound), dihydroartemissinin, and bilobalide (isolated from Ginkgo biloba).

Artemisinin is a sesquiterpene lactone drug used to treat multi-drug resistant strains of falciparum malaria. Artemisinin is isolated from the plant Artemisia annua, but can also be synthesized from artemisinic acid. Its structure is shown in FIG. 1L. Artemisinin is poorly soluble, which limits its bioavailability. Semi-synthetic derivatives of artemisinin, including artemether and artesunate, have been developed. However, their activity is not long-lasting, with significant decreases in effectiveness after one to two hours. To counter this drawback, artemisinin is given with lumefantrine (also known as benflumetol) to treat uncomplicated falciparum malaria. Lumefantrine has a half-life of about 3 to 6 days. Such a treatment is called ACT (artemisinin-based combination therapy); other examples are artemether-lumefantrine, artesunate-mefloquine, artesunate-amodiaquine, and artesunate-sulfadoxine/pyrimethamine. Recent trials have shown that ACT is more than 90% effective, with recovery from malaria after three days, even with chloroquine-resistant Plasmodium falciparum. A water solution of artemisinin would be highly desirable for direct parenteral applications.

Lignans.

Lignans are a class of compounds in which two phenylpropane coniferyl alcohol monomer units are coupled at the central carbon of the side-chain (lignans) or at another location (neolignans). Examples of lignans are podophyllotoxin (isolated from American Mayapple), 4′-demethylpodophyllotoxin, beta-peltatin, alpha-peltatin, desoxypodophyllotoxin, podophyllotoxone, matairesinol, yatein, and pinoresinol. Podophyllotoxin, also known as codylox or podofilox, is a lignan compound, and a non-alkaloid toxin isolated from the rhizome of American Mayapple (Podophyllum peltatum). Its structure is shown in FIG. 1M. Podophyllotoxin can also be synthesized biologically from two molecules of coniferyl alcohol. Podophyllotoxin is the pharmacological precursor for the important anti-cancer drug etoposide. It is also administered to treat genital warts. Podophylotoxin is poorly soluble in water, and a water solution containing a pharmaceutically effective amount has not been available.

Flavonolignans.

Flavonolignans are a class of compounds structurally combined from flavonoid and lignan. These include compounds such as silybin, isosilybin, and silychristin (seen in the plant of milk thistle (Silybum marianum) from the family of Compositae. Silybin, also known as Silibinin, is the major active constituent of silymarin, the mixture of flavonolignans extracted from milk thistle (Silybum marianum). The structure of silybin is shown in FIG. 1Q. Studies suggest that silybin has hepatoprotective (antihepatotoxic) properties and anti-cancer effects against human prostate adenocarcinoma cells, estrogen-dependent and estrogen-independent human breast carcinoma cells, human ectocervical carcinoma cells, human colon cancer cells, and both small and nonsmall human lung carcinoma cells. Poor water solubility and bioavailability of silymarin led to the development of enhanced formulations. Silipide (trade name SILIPHOS®), a complex of silymarin and phosphatidylcholine (lecithin), is about ten times more bioavailable than silymarin. It has been also reported that silymarin inclusion complex with β-cyclodextrin is much more soluble than silymarin itself. Glycosides of silybin show better water solubility and even stronger hepatoprotective effects. However, an aqueous solution of silybin in pharmaceutically acceptable amount, in its original and unmodified structure, has not been available for parenteral administrations.

Lipids.

Other water insoluble therapeutic compounds or mixtures of compounds include lipids, e.g. fatty acids in fish oil. Some of the beneficial components of fish oil (i.e., omega-3 fatty acids, including eicosapentaenoic acid and docosahexaenoic acid) are shown in FIG. 1R. Fish oil has been widely used as a neuroprotectant.

Azole. An azole is a class of five-membered nitrogen heterocyclic ring compounds containing at least one other noncarbon atom, for example, a nitrogen, sulfur or oxygen (Eicher, T.; Hauptmann, S. (2nd ed. 2003). The Chemistry of Heterocycles: Structure, Reactions, Syntheses, and Applications. Wiley-VCH. ISBN 3527307206). Itraconazole is a triazole with antifungal activities. The structure of itraconazole is shown in FIG. 1S. Other triazole antifungal drugs include fluconazole, isavuconazole, voriconazole, pramiconazole, posaconazole, ravuconazole, fluconazole, fosfluconazole, epoxiconazole, triadimenol, propiconazole, metconazole, cyproconazole, tebuconazole, flusilazole and paclobutrazol. These compounds are practically insoluble in water (e.g., itraconazole, The Merck Index, 1996, p. 895). Itraconazole has relatively low bioavailability after oral administration. Some improvement has been made, for example, in SPORANOX® using cyclodextrin complexation and propylene glycol to deliver the drug via intravenous infusion. True aqueous compositions of itraconazole have been limited by the poor water solubility.

Celecoxib is a pyrazole (a rare alkaloid), a compound that targets cyclooxygenase (COX) enzymes. The structure of celecoxib is shown in FIG. 1T. In medicine, pyrazoles are used for their analgesic, anti-inflammatory, antipyretic, antiarrhythmic, tranquilizing, muscle relaxing, psychoanaleptic, anticonvulsant, monoamineoxidase inhibiting, antidiabetic and antibacterial activities. Celecoxib is a COX-2 inhibitor. Celecoxib has poor solubility in water which reduces its bioavailability. True water solutions of celecoxib have not been reported.

All of the above and many other pharmaceutically active compounds are relatively insoluble in water. The potential use of these agents in therapy could be increased if the compounds could be made soluble in an aqueous solution.

Diterpene Glycosides

Natural terpene glycosides exist in a variety of plant sources. They generally are terpene aglycons attached to at least one glucose or other simple sugars (e.g., xylose or galactose), and the most common forms are monoterpene glycosides, diterpene glucosides, and triterpene glucosides. Many of these compounds are known to be non-toxic and natural sweeteners. (U.S. Published Patent Application No. 2006/0003053; and Chinese Patent No. 1723981). Examples of diterpene glycosides include rubusoside, rebaudioside, stevioside, and steviol monoside. Rubusoside A is a diterpene glycoside mainly from Chinese sweet leaf tea leaves (Rubus suavissimus; Rosaceae). Rubusoside A has a molecular formula C₃₂H₅₀O₁₃ and molecular weight of 642.73. The structure of rubusoside is shown in FIG. 2. (From T. Tanaka et al., Rubusoside (b-D-glucosyl ester of 13-O-b-D-glucosyl-steviol), a sweet principle of Rubus chingii Hu (Rosacease), Agricultural and Biological Chemistry, vol. 45(9), pp. 2165-6, 1981). Rubusoside also has good solubility in water, alcohol and acetone ethyl acetate. The compound as shown in FIG. 2 is a diterpene aglycone with two glucose molecules attached.

Another diterpene glycoside that is isolated from the Chinese sweet leaf tea (Rubus suavissimus; Rosaceae) and from stevia leaves (Stevia rebaudiana; Asteraceae) is steviol monoside. The structure of steviol monoside has only one glucose molecule (FIG. 5) rather than two as in rubusoside (FIG. 2). Steviol monoside can be isolated from the sweet leaf tea, stevia leaves, or be obtained through the partial acid or alkaline hydrolysis of rubusoside to cleave one glucose molecule. Unlike rubusoside, steviol monoside is not a dominant diterpene glycoside in the sweet leaf tea or stevia plant.

Stevioside is a diterpene glycoside that is isolated from the Stevia leaf (Stevia rebaudiana; Asteraceae). Stevioside has a molecular formula C₃₈H₆₀O₁₈ and a molecular weight of 804. The structure is shown in FIG. 3. The compound as shown is a diterpene aglycone with three glucose molecules. In pure form, it is a crystal or white powder. Another diterpene glycoside that is isolated from the Stevia leaf is rebaudioside, which exists in several forms, including rebaudioside A, rebaudioside B, rebaudioside C, rebaudioside D, rebaudioside E, and rebaudioside F. The structure of rebaudioside A is shown in FIG. 4. The compound as shown is a diterpene aglycone with four glucose molecules. In pure form, it is a white powder.

Other diterpene that contain various numbers of glucose moieties have been described. These compounds include: paniculoside IV, suaviosides A, B, C₁, D₁, D₂, E, F, G, H, I, and J (FIG. 5) as identified by Ohtani et al. (1992, Phytochemistry 31(5): 1553-1559), and goshonosides F₁ to F₅ (FIG. 6) as identified by Seto et al. (1984, Phytochemistry 23 (12): 2829-2834). Although many diterpene glycosides such as stevioside, rebaudioside A, rubusoside, steviol monoside, and suavioside B, G, I, J, and H taste sweet, other diterpene glycosides are tasteless or bitter. For examples, paniculoside IV is tasteless, suavioside C₁ tastes bitter, suavioside D₁ is tasteless, suavioside D₂ tastes bitter, suavioside E is tasteless, and suavioside F tastes bitter as indicated by Ohtani et al. (1992, Phytochemistry 31(5): 1553-1559).

Permeability Glycoprotein (P-gp)

P-gp is extensively distributed and expressed in the intestinal epithelium, hepatocytes, renal proximal tubular cells, adrenal gland and capillary endothelial cells comprising the blood-brain and blood-testis barrier. P-gp is an ATP-dependent drug efflux pump for xenobiotic compounds with broad substrate specificity. It is responsible for decreased drug accumulation in multidrug-resistant cells and often mediates the development of resistance to anticancer drugs. This protein also functions as a transporter in the blood-brain barrier.

Many substrate drugs of P-gp have low gastrointestinal absorption thus low oral bioavailability Inhibiting P-gp can increase drug absorption in the intestines, which increases oral bioavailability. Many drugs such a digoxin, paclitaxel, etoposide are substrates of P-pg, thus intestinal absorption into the blood stream is reduced as a result of efflux pumping activities. Current P-gp inhibitors, e.g., cyclosporine and ritonavir, are effective, but can cause harmful side effects. For example, cyclosporine is an immune suppressant and its use to inhibit P-gp also decreases the immune function. Ritonavir is an antiretroviral drug compound from the protease inhibitors class, and its use to inhibit Pgp causes unwanted and unnecessary physiological responses.

U.S. Published Patent Application No. 2002/0076426 discloses terpene alcohol ethoxylates as solubilizers in pharmaceutical and food preparations.

Chinese Patent No. 1723981 discloses that an extract containing triterpene glycosides (mogrosides) isolated from Momordica grosvenoiri fruit was used to replace sucrose or other sweeteners in manufacturing pills, granules, tablets, capsules or solutions of traditional Chinese medicine.

I have previously shown that diterpene glycosides are effective solubilizers for many classes of organic compounds that are insoluble or sparingly soluble in aqueous solution. See, International Patent Application No. PCT/US2009/040324, now published as International Published Application No. WO 2009/126950, incorporated completely into this provisional application.

### DISCLOSURE OF INVENTION

I have discovered a method to produce a powder form of a compound-solubilizer complex than can be dissolved in water. The compounds, usually drugs, were insoluble or sparingly soluble in water, including some fat-insoluble compounds (e.g., fat-soluble vitamins), and were dissolved by a diterpene solubilizer, for example, rubusoside. The compound-solubilizer complex was then dehydrated to a stable powder that was reconstituted without destroying the drug effectiveness. These powders were made using both rebusoside and rebaudioside A as the solubilizers, and could be made using other diterpene glycoside solubilizers. The powder form has many advantages over a liquid form for storage and administration. I also developed a more effective process of making the initial compound-solubilizer complex that dissolved more compound in water using the solublizer. In addition, I have shown that a diterpene glycoside, rubusoside, is an inhibitor of permeability glycoprotein (P-gp), and will thus increase gastrointestinal absorption of compounds administered with rubusoside.

### BRIEF DESCRIPTION OF DRAWINGS

FIGS. 1A to 1V illustrates the structures of representative compounds of several classes of compounds that are known to have low water solubility, and that have been shown to be solubilized using a diterpene glycoside, including gallic acid (FIG. 1A), rutin (FIG. 1B), tanshinone IIA (FIG. 1C), Co-Q10 (FIG. 1D), curcumin (FIG. 1E), camptothecin (FIG. 1F), capsaicin (FIG. 1G), paclitaxel (FIG. 1H), cyclosporin A (FIG. 1I), erythromycin (FIG. 1J), nystatin (FIG. 1K), artemisinin (FIG. 1L), podophyllotoxin (FIG. 1M), alpha-tocopherol (FIG. 1N), propofol (FIG. 1O), 6-gingerol (FIG. 1P), silybin (FIG. 1Q), omega-3 fatty acids (eicosapentaenoic acid and docosahexaenoic acid) (FIG. 1R), itraconazole (FIG. 1S), celecoxib (FIG. 1T), rapamycin (FIG. 1U), and amphotericin B (FIG. 1V).

FIG. 2 illustrates the structure of rubusoside, a diterpene glycoside isolated from Chinese sweet leaf tea.

FIG. 3 illustrates the structure of stevioside, a diterpene glycoside isolated from the Stevia leaf.

FIG. 4 illustrates the structure of rebaudioside A, another diterpene glycoside isolated from Stevia leaf.

FIG. 5 illustrates the structures of several diterpene glycosides isolated from Rubus or Stevia plants.

FIG. 6 illustrates the structures of several diterpene glucosides isolated from Rubus or Stevia plants.

FIG. 7 illustrates the results of high performance liquid chromatography indicating the amount of curcumin dissolved in 5% rubusoside water solution using the Standard process (lower) and the Enhanced process (upper).

FIG. 8 illustrates the linear relationship between the rubusoside concentration and the amount of curcumin dissolved in the rubusoside water solution.

FIG. 9 illustrates the results of high performance liquid chromatography indicating curcumin dissolved in 5% w/v rebaudioside A water solution with the upper chromatogram indicating the amount of rebaudioside A and the lower chromatogram the amount of curcumin.

FIG. 10 illustrates the results of high performance liquid chromatography indicating the amount of paclitaxel dissolved in 10% rubusoside water solution using the Standard process (lower) and the Enhanced process (upper).

FIG. 11 illustrates the results of high performance liquid chromatography indicating the amount of camptothecin dissolved in 10% rubusoside water solution using the Standard process (lower) and the Enhanced process (upper).

FIG. 12 illustrates a curcumin and 30% rubusoside complex both in powder form and in reconstituted form.

FIG. 13 illustrates the results of high performance liquid chromatography indicating the amount of curcumin and rubusoside in a reconstituted solution.

FIG. 14 illustrates the effect of various concentrations of a curcumin-rubusoside solution after reconstitution on the growth of human pancreatic cancer cells (Panc-1).

FIG. 15 illustrates the results of high performance liquid chromatography indicating the amount of paclitaxel dissolved in 10% rubusoside water solution, then dried to powder, and reconstituted using various amounts of water and diluted to the original concentration if necessary.

FIG. 16 illustrates the results of high performance liquid chromatography indicating the amount of four fat-soluble vitamins (A, D, E, and K) dissolved in a 10% rubusoside solution.

FIG. 17 illustrates the results of high performance liquid chromatography of a mixed vitamin water solution containing the fat-soluble vitamins A, D, E, and K in the presence of 10% rubusoside (Upper), as compared to similar mixture in methanol (Lower).

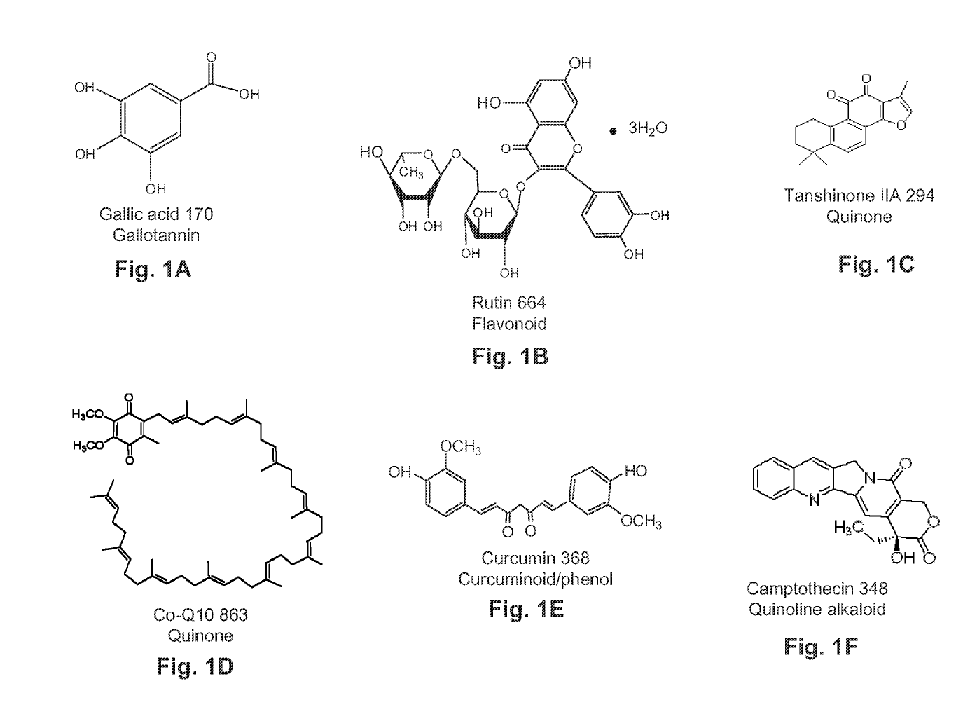

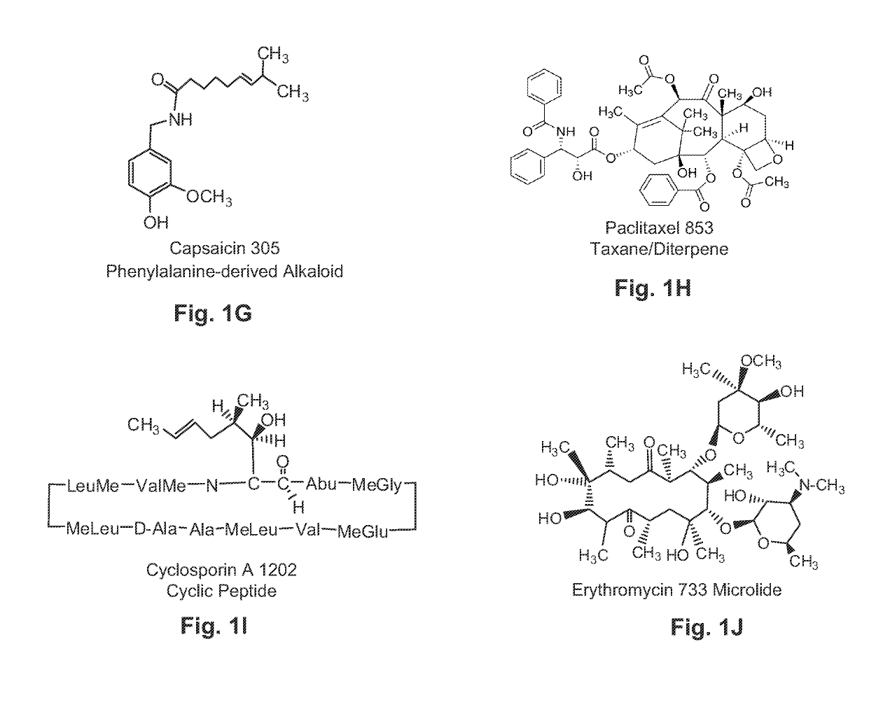

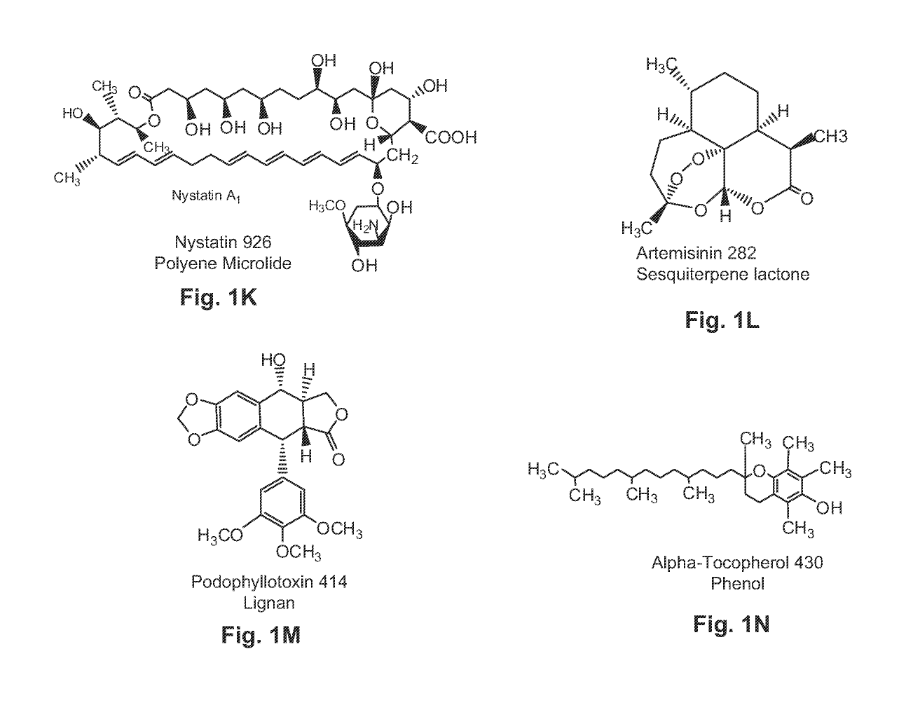

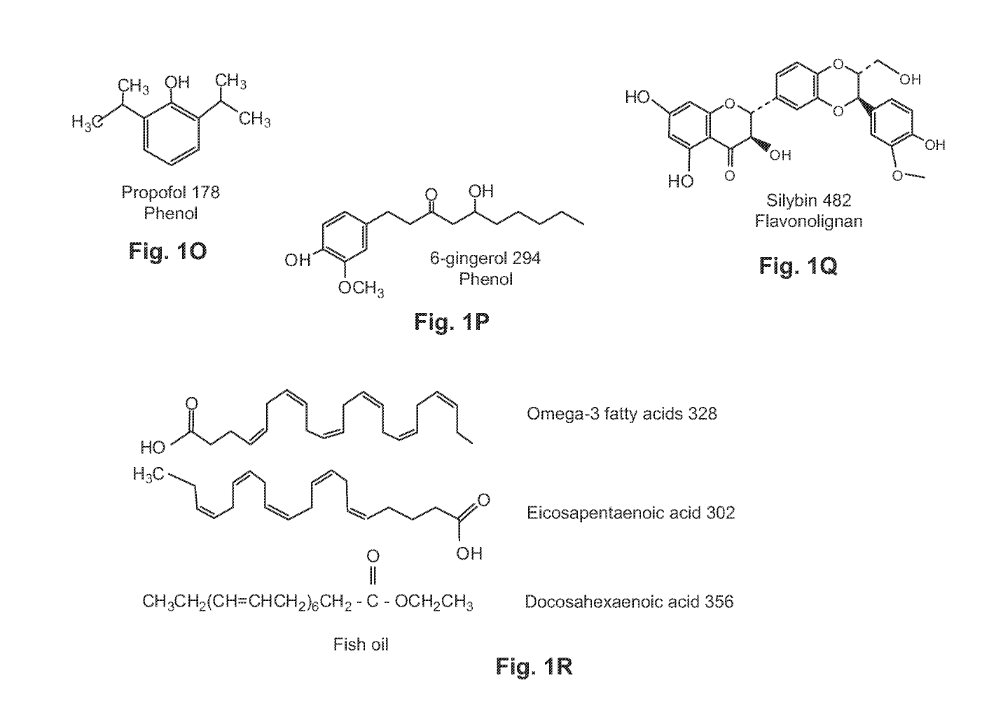

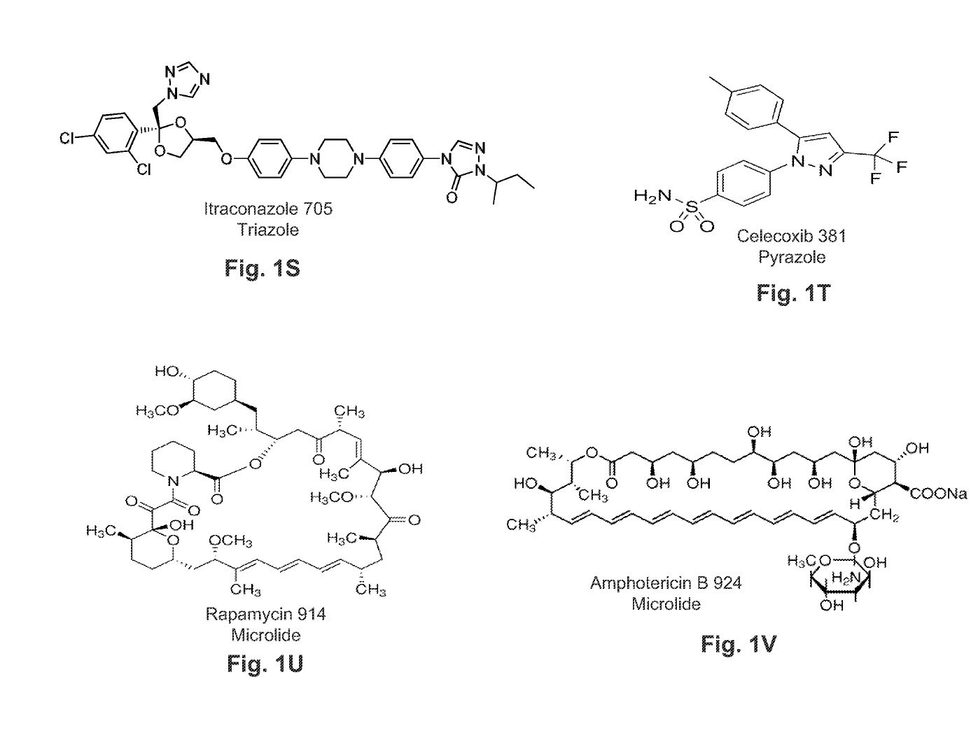

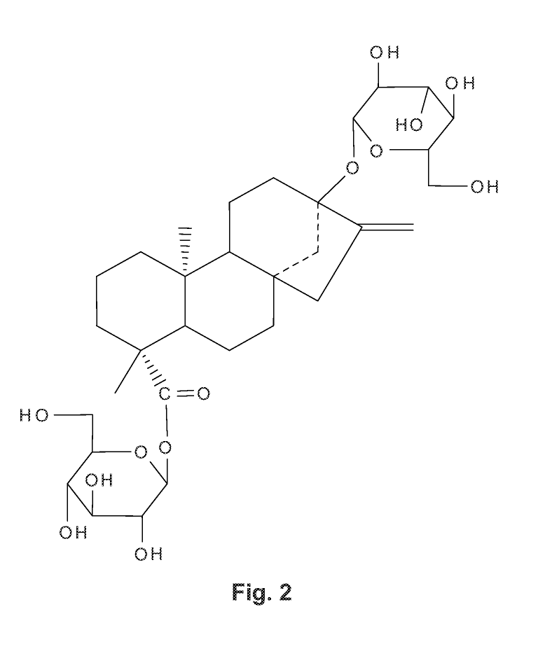

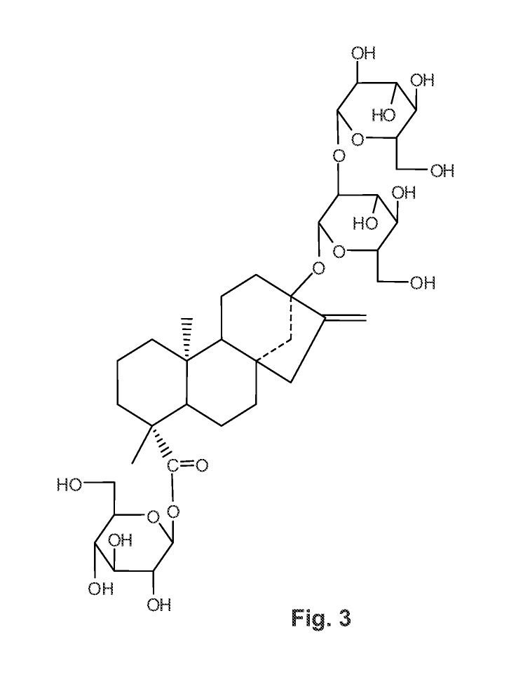

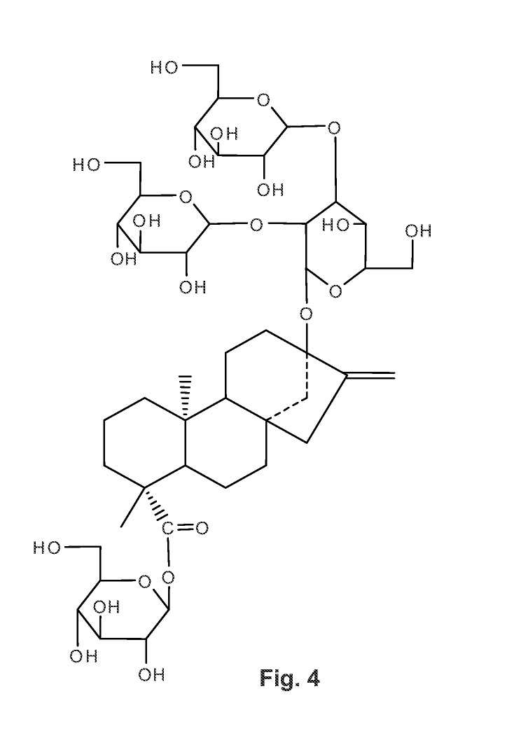

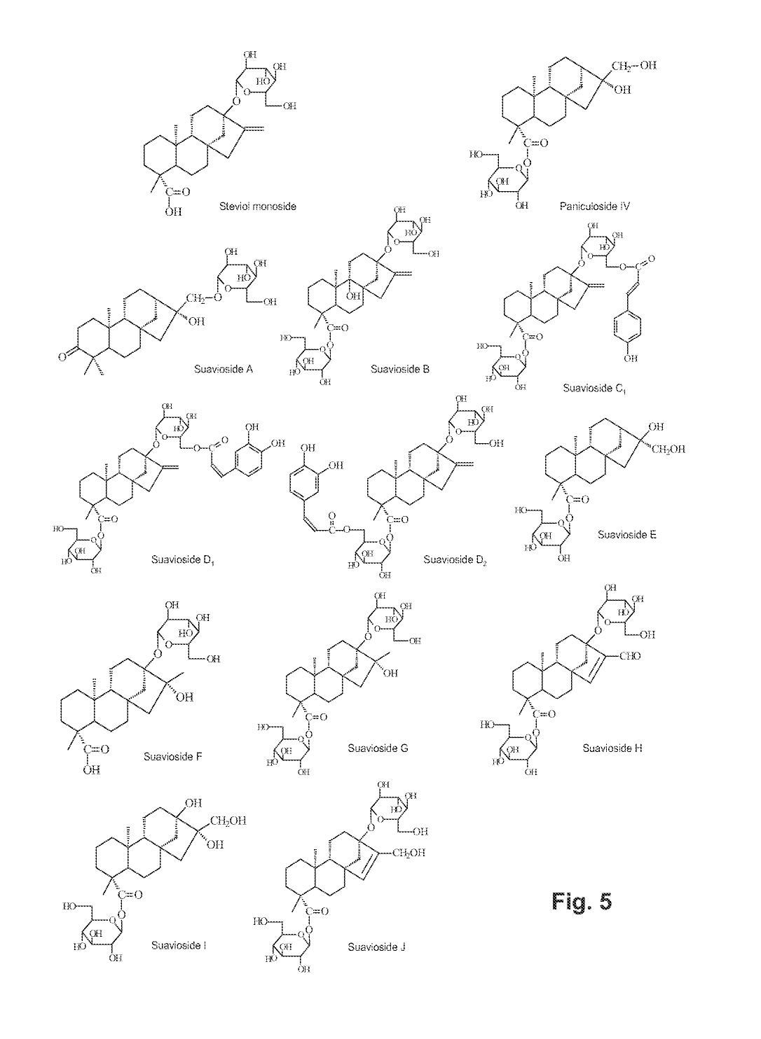

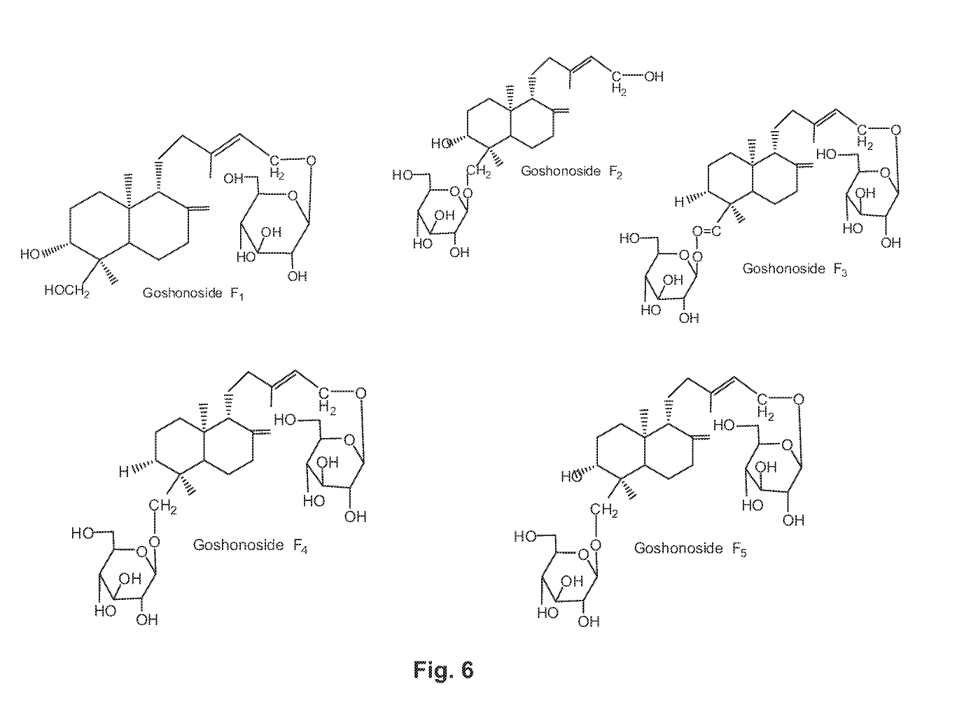

### MODES FOR CARRYING OUT THE INVENTION

Several important organic compounds are insoluble in water or have very low solubility. I have previously tested many of these therapeutic compounds from several classes of chemical structures and found that natural solubilizers based on diterpene glycosides have increased the aqueous solubility of all compounds tested. (See, WO 2009/126950) I previously found a method for enhancing the solubility of an organic compound which is insoluble or sparingly soluble in water, said method comprising mixing said compound with water and with a diterpene glycoside in a concentration sufficient to increase the solubility of the compound in water by a factor of 2 or more. The solubility for the organic compounds in some cases has been increased by a factor of 5 or more, in others by a factor of 10 or more, in others by a factor of 20 or more, in others by a factor of 50 or more, in others by a factor of 100 or more, and in others by a factor of 1000 or more. I have now discovered that diterpene glycosides can solubilize fat-soluble compounds, including the fat-soluble vitamins.

I have discovered a method to make the drug-solubilizer complex a powder. The powder was shown to be stable, and can be reconstituted. The drug effectiveness was not affected by the dehydration and reconstitution procedure.

I have discovered diterpene glycosides as new solubilizing agents for creating new powdered formulations that will be useful in pharmaceutical, cosmetic, agricultural and food formulations instead of the commonly used cyclodextrins.

Using the diterpene glycosides as solubilizers provides a way to alleviate problems with low solubility drugs, e.g., low absorption and low bio-availability of the drug. Rubusoside was discovered to inhibit permeability glycoprotein which will improve gastrointestinal absorption. In addition, using the solubilizer and drug in a powder form (containing solubilizer-drug complexes) will allow solid formulations that are readily dissolvable in water, e.g., tablet or even effervescent tablets. The solubilizers can be used to prepare non-alcoholic syrups of low solubility drugs that are stable, or to prepare gelatin capsules with the solubilizer and drug inside.

The powdered form of the solubilizer and solubilized drug may be administered to a patient by any suitable means, either as a powder or a reconstituted liquid, including orally, parenteral, subcutaneous, intrapulmonary, topically (e.g., ocular or dermal), rectal and intranasal administration. Parenteral infusions include intramuscular, intravenous, intraarterial, or intraperitoneal administration. The solution or its dry ingredients (containing solubilizer-drug complexes) may also be administered transdermally, for example in the form of a slow-release subcutaneous implant, or orally in the form of capsules, powders, or granules.

Pharmaceutically acceptable carrier preparations for parenteral administration include sterile, aqueous or non-aqueous solutions, suspensions, and emulsions. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The solubilizer and drug may be mixed with other excipients that are pharmaceutically acceptable and are compatible with the active ingredient in the drug. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

For purposes of this application, a compound that is insoluble in water is a compound in which less than 100 μg dissolves in 1 mL water. A compound that is sparingly soluble in water is one in which less than 20 mg, but more than 100 μg, dissolves in 1 mL water. Finally, in general, a compound that has low solubility in water is one in which less than 20 mg dissolves in 1 mL water.

### Formation of Powdered Drug-Solubilizer Complexes

### Example 1

Materials and Methods

Sonication-Autoclave, an Enhanced Process for Solubilizing.

A new method (the “Sonication—autoclave method”) was used to solublize most of the compounds, a method different from that reported in International Patent Application No. PCT/US2009/040324 (the “Standard Shake Flask method”). The main difference was the use of higher temperatures and elevated atmospheric pressures. The steps of the new enhanced process are as follows. A water-insoluble compound was weighed into multiple flasks, divided into two groups—a control group and an experimental group. Each experimental flask received a known amount of the solubilizing agent, e.g. rubusoside, being tested. The control flasks contained only the water-insoluble compound. The same volume, 10 mL, unless otherwise indicated, of deionized and distilled water was added to each flask. Alternatively, water solutions containing a known percentage of solubilizer (e.g., 5% or 10% w/v) were prepared. The solubilizer-water solutions were added directly to the experimental flasks containing the water-insoluble compound. In either case, the flasks were then vortexed briefly and sonicated for 60 min at 50° C. Sonication is a process of applying sound (usually ultrasound) energy to agitate particles in a sample. Using this energy, inter-molecular interactions are facilitated. After sonication, the solution was then subjected to heat and pressure. The heat and pressure treatment was composed of either 121° C. and 1.1 to 1.2 atm (standard atmospheric pressure) or 134° C. and 2 to 2.1 atm, similar to those used in a standard sterilization procedure in an autoclave, e.g., in a Tuttnauer 3870M Analog Autoclave. The length of heat and pressure was set from about 30 min to about 60 min. The heat can be increased to even higher temperatures as long as the structure of the water-insoluble compound or the solubilizer does not irrevocably change. After 30 min of heat and pressure treatment, the flasks were placed in an incubator set at 25° C. for at least 24 hr. The solutions were then centrifuged at 4000 rpm for 10 min. The supernatant solution was passed through a 0.45 μm filter and analyzed for the concentration of the water-insoluble compound or solubilizer by HPLC or LC/MS, as described below.

Homogenization-Autoclave, Another Enhanced Process for Solubilizing

In another method (Homogenization-Autoclave Method), a water-insoluble compound was weighed into a flask containing a known amount of the solubilizing agent, e.g. rubusoside. Homogenization is a process of mechanically blending ingredients together in uniform distribution. Homogenization facilitates the inter-molecular interactions for form complex structures. The same volume, 10 mL, unless otherwise indicated, of deionized and distilled water was added to the flask. Alternatively, water solutions containing a known percentage of solubilizer (e.g., 5% or 10% w/v) were prepared. The solubilizer-water solution was added directly to an experimental flask containing the water-insoluble compound. In either case, the flask was then homogenized (Cyclone I.Q.2 homogenizer, Virtis Corp) at a single speed between about 4,000 rotation per minute (rpm) and about 22,000 rpm for a period of time from about 30 seconds to about 20 min. In the case of curcumin, the speed was 22,000 rpm for 1 min. The speed and time was chosen for each drug. The solution was then subjected to heat and pressure. The heat and pressure treatment was composed of either 121° C. and 1.1 to 1.2 atm (standard atmospheric pressure) or 134° C. and 2 to 2.1 atm, similar to those used in a standard sterilization procedure in an autoclave, e.g., in a Tuttnauer 3870M Analog Autoclave. The length of heat and pressure was set from about 30 min to about 60 min. The heat can be increased to even higher temperatures as long as the structure of the water-insoluble compound or the solubilizer does not irrevocably change. After 60 min of heat and pressure treatment, the flasks were placed in an incubator set at 25° C. for at least 24 hr. The solutions were then centrifuged at 4000 rpm for 10 min. The supernatant solution was passed through a 0.45 μm filter and analyzed for the concentration of the water-insoluble compound or solubilizer by HPLC or LC/MS, as described below. Results in curcumin concentrations from different processing methods are listed in the following table, Table 1.

TABLE 1
Comparison of processing method on curcumin concentration

| | | Solubi- | Curcumin |
|---|---|---|---|
| | | lizer | concen- |
| Processing | | content | tration |
| method name | Processing method features | (w/v) | (mg/mL) |
| Shake-flask | Shaking at a speed of 80 rmp | 10% | 0.34 |
| Standard Method, | for 24 hr at 25° C. | | |
| as described in | | | |
| WO 2009/126950 | | | |
| Sonication- | Sonication then autoclave, | 10% | 1.3 |
| Autoclave | followed by shaking at a speed | | |
| Enhanced Method | of 80 rmp for 24 hr at 25° C. | | |
| Homogenization- | Homogenization then | 10% | 2.4 |
| autoclave | autoclave, followed by shaking | | |
| Enhanced | at a speed of 80 rmp for 24 hr | | |
| Method | at 25° C. | | |

As shown in Table 1, the Homogenization-Autoclave Method was more effective in solubilizing curcumin than the other two methods. This method has been tried with other drugs, including paclitaxel (See Example 3), rutin (See Example 5), and cyclosporine (250 μg/mL by the standard method, 662 μg/mL by the sonication-autoclave method, and 1322 μg/mL by the homogenization-autoclave method (at a speed of 8000 rpm for 5 min)). It has also been tried with other solubilizers, such as rebaudioside A. It has shown to be the best method to solubilize all compounds tried, and is believed to be the best method to solubilize all compounds with the diterpene glycosides.

HPLC-UV and HPLC-MS Analysis: The solutions containing various water-insoluble compounds in the absence or presence of solubilizers were analyzed, unless otherwise indicated, on HPLC-UV or HPLC-MS which consisted of a solvent delivery pump unit, an autosampler (Waters 717 plus), a UV-Vis diode array detector (Waters 2996 Photodiode Array Detector, 190 to 800 nm) coupled with an EMD 1000 Mass Detector (Waters), and an evaporative light-scattering detector (Waters 2420 ELSD). The system was computer controlled, and the results were analyzed using Empower software. Calibration curves were constructed using known concentrations of the compounds and were used to quantify the concentrations of the compounds dissolved in solution.

Powders Containing Water Soluble Complexes of Solubilizer and Water-Insoluble Compound.

The water solutions containing the complexes of the water-insoluble compound and the solubilizer were dried to remove the water and to form a powder. The powder contained at minimum about 1% w/w water. Any method of water removal could be used, including freeze-drying, spray-drying, or oven drying. The powder was then shown to be able to be re-constituted in water so that the water-insoluble compound remained in solution, as a complex with the solublizer. The amount of added water can be adjusted to achieve the desired concentration of the compound.

Rebaudioside A has also been used as a solubilizer with curcumin, progesterone, and resveratrol, and the solution dried to a powder and successfully reconstituted. Without wishing to be bound by this theory, it is believed that other diterpene glycosides would also be able to form a drug-solubilizer complex in solution with an organic compound that has low solubility in water, to maintain that complex upon drying the solution to a powder, and to be reconstituted in a solution upon addition of water to the powder. PCT/US2009/040324, contains a list of other diterpene glycosides and a list of organic compounds with low solubility in water that could be used to make the powder and solution.

### Example 2

Effect of the Processing Method on the Water Solubility of Curcumin.

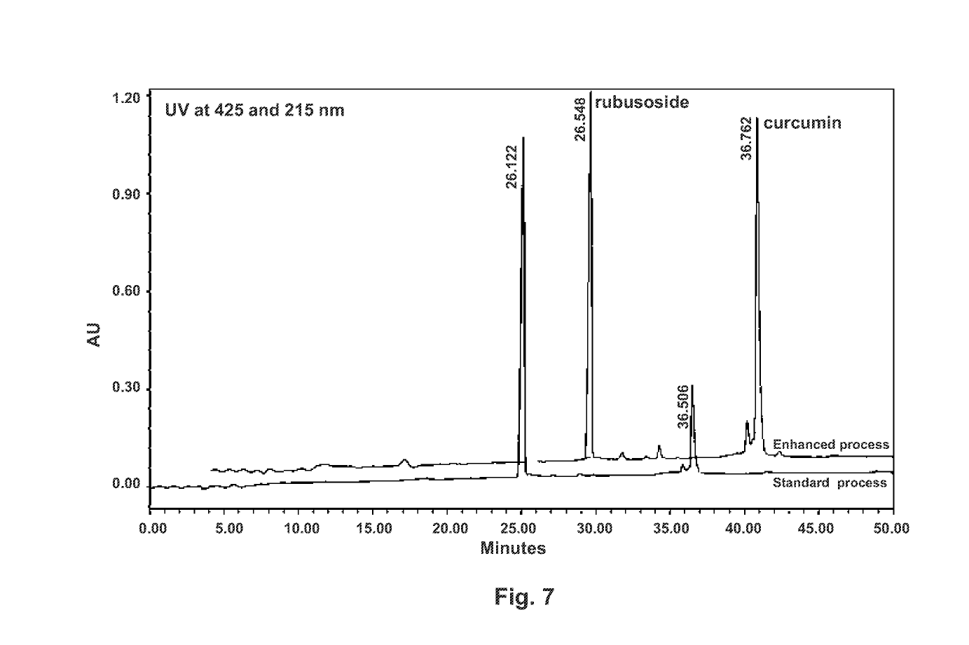

5% w/v of rubusoside water solution was prepared. One hundred milligrams of curcumin was weighed into separate flasks. One solution was processed based on the “standard” processing method as described in International Patent Application No. PCT/US2009/040324 (“standard process”) and the other based on the new “enhanced” method using elevated heat and atmospheric pressure (“enhanced process”). The curcumin-rubusoside solutions were filtered using a 0.45 μM filter and analyzed on HPLC. Quantification was done by comparing to a standard solution of a known amount of curcumin in methanol. FIG. 7 shows chromatograms of curcumin water solutions containing 5% w/v rubusoside as a natural solubilizer prepared using the standard process and the enhanced process. The HPLC (Waters HPLC system with 600 pump, 717 autosampler, and 2996 PDA) chromatograms were generated using a Phenomenex luna C18 column (4.6×250 mm, 5 μm) and a mobile phase of 0.02% HCOOH-ACN (A): 0.02% HCOOH—H2O (B), the gradient was A from 20% to 80% in 45 min; at a flow rate of 1.0 mL/min, injection volume of 5 μL, UV detection dual wavelengths of 425 nm and 215 nm, and column temperature of 30 C. The chromatograms of curcumin solutions were generated at 425 and 215 nm UV showing elution of rubusoside at 25.548 min and curcumin at 36.752 min. In the presence of 5% w/v of rubusoside, the standard process produced a solution containing 150 μg/mL curcumin. In contrast, the enhanced process produced a solution containing 622 μg/mL curcumin (FIG. 7), more than 4-fold increase.

To develop a dose response, a series of rubusoside water solutions were prepared ranging from 1% w/v to 40% w/v. Curcumin was added to each solution using the enhanced process as described above. Curcumin concentrations in each water solution were analyzed on HPLC. As Table 2 shows, without rubusoside, the enhanced process dissolved undetectable curcumin whereas 1%, 2.5%, 5%, 10%, 20%, 30%, and 40% rubusoside water solutions dissolved 28, 267, 762, 1330, 2914, 4449, and 6004 μg/mL curcumin, respectively.

TABLE 2
Solubility of curcumin in various concentrations of rubusoside

| Rubusoside | Curcumin in water |
|---|---|
| (%, w/v) | Solution (μg/mL) |
| 0 | 0 |
| 1.0 | 28 |
| 2.5 | 267 |
| 5.0 | 762 |
| 10.0 | 1330 |
| 20.0 | 2914 |
| 30.0 | 4449 |
| 40.0 | 6004 |

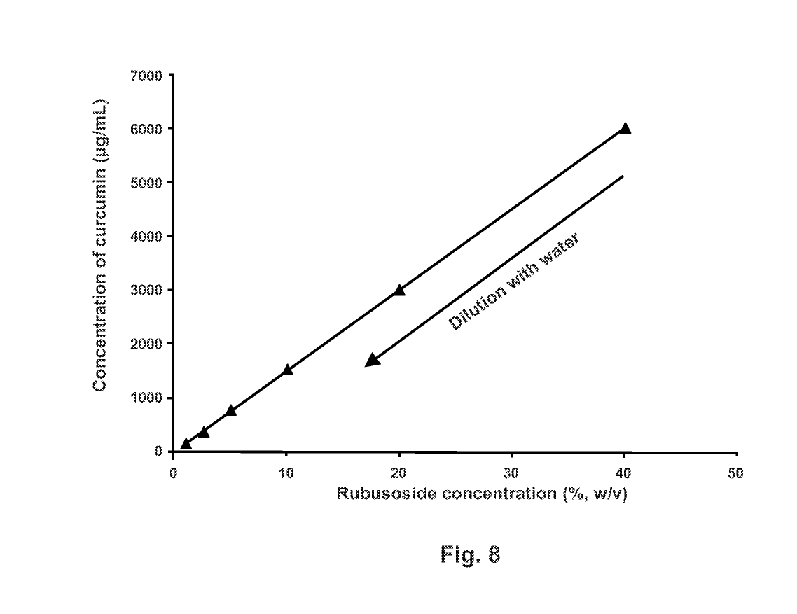

To test if the most concentrated curcumin solution could be diluted with water, the 40% curcumin water solution containing 6004 μg/mL curcumin was diluted by 1, 2, 4, 8, 16, and 40 fold with water, and the diluted water solutions were analyzed on HPLC for curcumin concentrations. As Table 3 and FIG. 8 show, dilutions resulted in a linear decrease of curcumin concentrations. No precipitation of either rubusoside or curcumin was observed for at least one week. This shows the stability of curcumin-rubusoside solution when diluted with water. This is important because the concentrated curcumin solution may be diluted in a biological system, e.g., in the stomach fluid.

TABLE 3
Results of dilution of a concentrated curcumin-rubusoside solution

| Dilution | Rubusoside | Curcumin in water |
|---|---|---|
| factor | concentration (%, w/v) | Solution (μg/mL) |
| Stock | 40.0 | 6004 |
| 1:1 | 20.0 | 2985 |
| 1:3 | 10.0 | 1489 |
| 1:7 | 5.0 | 742 |
| 1:15 | 2.5 | 358 |
| 1:39 | 1.0 | 135 |

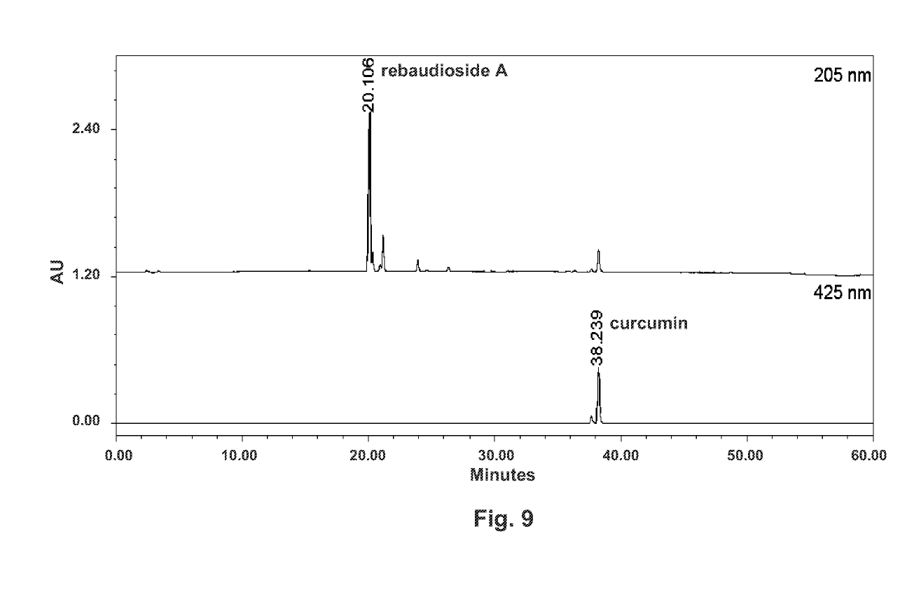

To test if the enhanced process also applies to another solubilizing agent, rebuadioside A was used. A 5% w/v of rebaudioside A water solution was prepared. One hundred milligrams of curcumin was weighed into separate flasks. One solution was treated using the standard process, and the other based on the enhanced process. The water solutions were filtered by 0.45 μM filters and analyzed on HPLC. Quantification was done by comparing to a standard solution of a known amount of curcumin in methanol. In FIG. 9 are shown chromatograms of curcumin solutions containing 5% w/v rebaudioside A as a solubilizing agent. The HPLC (Waters HPLC system with 600 pump, 717 autosampler, and 2996 PDA) Chromatograms were generated using a prevail C18 column (4.6×250 mm, 5 μm) and a mobile phase of 0.02% HCOOH-ACN (A): 0.02% HCOOH—H2O (B); the gradient was A from 20% to 80% in 45 min at a flow rate of 1.0 mL/min; the injection volume was 2 μL; UV detection wavelengths were 205 and 425 nm; and column temperature was 30 C. Curcumin concentration was determined using a standard curcumin solution of 196.8 μg/mL. The chromatograms in FIG. 9 were generated at 205 and 425 nm UV showing elution of curcumin at 38.239 min and rebaudioside A at 20.106 min. In the presence of 5% w/v of rubusoside, the standard process produced a water solution containing 156 μg/mL curcumin, and the enhanced process produced a water solution containing 302 μg/mL curcumin (FIG. 9), almost a 2-fold increase.

### Example 3

Effect of the Processing Method on the Water Solubility of Paclitaxel.

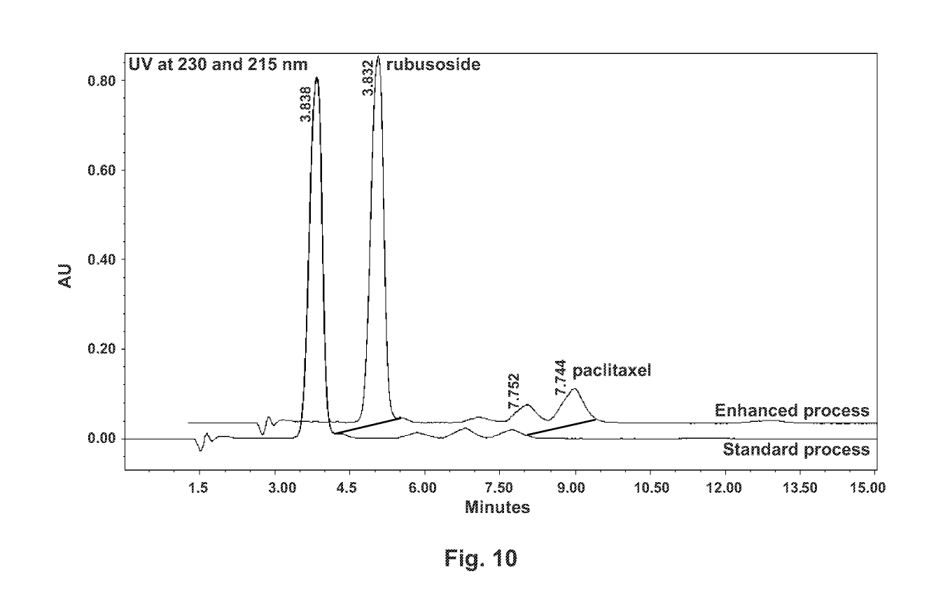

A 10% w/v of rubusoside water solution was prepared. Five milligrams of paclitaxel was weighed into separate flasks, and then 10 mL of the rubusoside solution was added to each. One solution was treated by the standard process, and the other using the enhanced process. The water solutions were filtered by 0.45 μM filters and analyzed on HPLC. Quantification was done by comparing to a standard solution of a known amount of either paclitaxel or rubusoside in methanol. FIG. 10 shows chromatograms of paclitaxel water solutions containing 10% w/v rubusoside as a natural solubilizer prepared using the Shake-flask Standard Method (“Standard process”) and the Sonication-Autoclave Enhanced Method (“Enhanced process”). The HPLC-MS (Waters HPLC-MS system with 600 pump, 717 autosampler, 2996 PDA, and an EMD1000 MS detector) chromatograms were generated using a Prevail C18 column (2.1×150 mm, 3 μm) and a mobile phase of 0.25% HCOOH:ACN:MeOH (4:4:2 v/v/v); at a flow rate of 0.40 mL/min, injection volume of 1 μL, UV detection wavelength of 230 nm, and column temperature of 30 C. MS detection was performed with MS-ESI in positive mode; a full scan range of m/z 200-960, and a SIR scan at m/z 854.4. The chromatograms were generated at 230 nm and 215 nm UV showing elution of rubusoside at 3.832 min and paclitaxel at 7.744 min. In the presence of 10% w/v of rubusoside, the standard process produced a water solution containing 65 μg/mL paclitaxel, and the enhanced process produced a water solution containing 269 μg/mL paclitaxel (FIG. 10), a more than 4-fold increase. In another experiment, the Homogenization-autoclave Enhanced Method was used at a speed of 8000 rpm for 5 min, and 725 μg/mL paclitaxel was dissolved in solution. (Chromatogram not shown) This is more than an 11-fold increase as compared to the Shake-flask Method, and an almost 3-fold increase as compared to the Homogenization-autoclave method.

### Example 4

Effect of the Processing Method on the Water Solubility of Camptothecin.

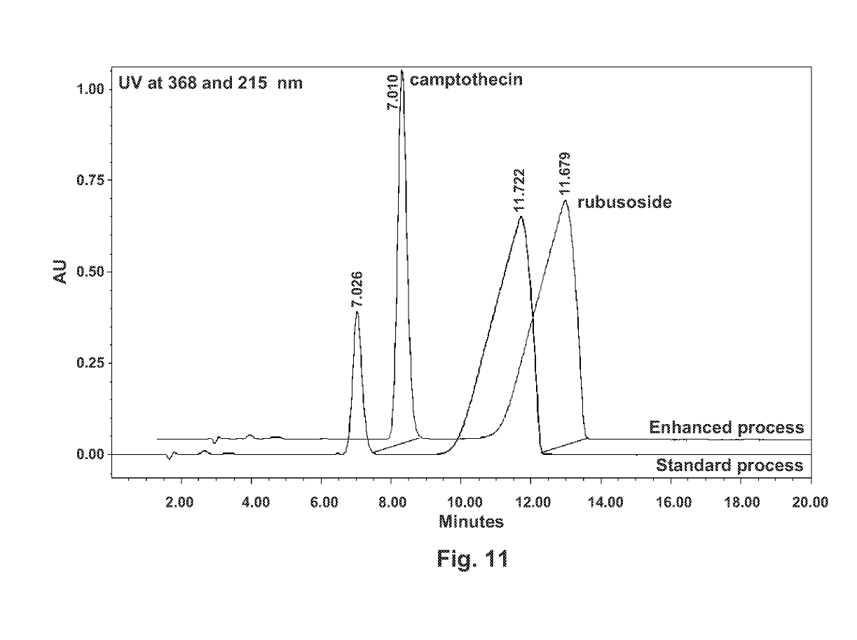

A 10% w/v of rubusoside water solution was prepared. Five hundred milligrams of camptothecin was weighed into separate flasks and then 10 mL rubusoside solution was added to each. One solution was processed based on the standard process and the other based on the enhanced process. The water solutions were filtered by 0.45 μM filters and analyzed on HPLC. Quantification was done by comparing to a standard solution of a known amount in methanol. FIG. 11 shows chromatograms of camptothecin water solutions containing 10% w/v rubusoside as a natural solubilizer prepared using the standard process and the enhanced process. The HPLC system included 600 pump, 717 autosampler, and 2996 PDA. Chromatograms were generated using a Prevail C18 column (2.1×150 mm, 3 μm) and a mobile phase of ACN (32): 0.02% HCOOH—H2O (68) at a flow rate of 0.4 mL/min, injection volume of 1 μL, UV detection wavelength of 368 nm, and column temperature of 30 C. The chromatograms in FIG. 11 were generated at a dual wavelength of 368 nm and 215 nm UV showing elution of camptothecin at 7.010 min and rubusoside at 11.679 min. In the presence of 10% w/v of rubusoside, the standard process produced a water solution containing 80 μg/mL camptothecin, and the enhanced process produced a water solution containing 247 μg/mL camptothecin (FIG. 11), a more than 3-fold increase.

### Example 5

Effect of the Processing Method on the Water Solubility of Rutin.

A 1% w/v of rubusoside water solution was prepared. Five milligrams of rutin was weighed into a separate flask, and then 10 mL of the 1% rubusoside solution was added. Using the Homogenization-autoclave Method, the water solution was homogenized (Virtis homogenizer) at a single speed of 20,000 rpm for 1 min. The solution was then subjected to heat and pressure. The heat and pressure treatment was a temperature of about 121° C. and a pressure of about 1.1 to 1.2 atm (standard atmospheric pressure), similar to those used in a standard sterilization procedure in an autoclave, e.g., in a Tuttnauer 3870M Analog Autoclave. The length of heat and pressure was set for 60 min. After 60 min of heat and pressure treatment, the flask was placed in an incubator set at 25° C. for 24 hr. The solution was then centrifuged at 4000 rpm for 10 min. The supernatant solution was passed through a 0.45 μm filter and analyzed for the concentration of rutin by HPLC. (Data not shown) The standard process (Shake-flask Method) produced a water solution containing 1.75 mg/mL rutin by 10% rubusoside, and the Homogenization-autoclave Method produced a water solution containing 2.7 mg/mL rutin with the use of only 1% rubusoside.

### Example 6

Formation of Curcumin-Solubilizer Complexes in Dried Powder and its Re-Constitution to Water Solutions

Various concentrations of the solubilizer rubusoside were prepared in water: 0%, 1%, 2.5%, 5%, 10%, 20%, 30%, and 40% w/v. Curcumin (100 mg) was weighed and added into separate flasks, and an equal amount (10 ml) of a rubusoside solution was added to each flask. The prepared water solutions were vortexed briefly and then sonicated for 60 min at temperature of 50° C. These water solutions were subjected to a heat and pressure treatment composed of 121° C. and 1.1 to 1.2 atm for 60 min, and then placed in an incubator set at 25° C. for at least 24 hr. The solutions were then centrifuged at 4000 rpm for 10 min. The supernatant solution was passed through a 0.45 μm filter and analyzed for the concentration of either the water-insoluble compound or rubusoside by HPLC or LC/MS, as described above in Example 2. Quantification was done by comparing to a standard solution of a known amount in methanol. In the presence of 0%, 1%, 2.5%, 5%, 10%, 20%, 30%, and 40% w/v of rubusoside, the water solutions contained 0, 28, 267, 762, 1330, 2914, 4449, and 6004 μg/mL curcumin, respectively. The color of the solutions using curcumin in 0%, 1%, 2.5%, 5%, 10% w/v rubusoside solutions was observed to become more a deep yellow color as the concentration of curcumin and rubusoside increased. (Data not shown) These curcumin-rubusoside water solutions were stable when kept in the dark at room temperature and at a pH from 4.5 to 5.2 for at least six weeks, based on HPLC analyses. When adjusted to about pH 7.0 by the addition of 1× phosphate buffered saline (PBS) powder, the solutions remained stable for at least 48 hours. Thus the reconstituted solution with saline would be sufficiently stable at pH 7.0 for use in a clinic setting.

Powder could be Reconstituted.

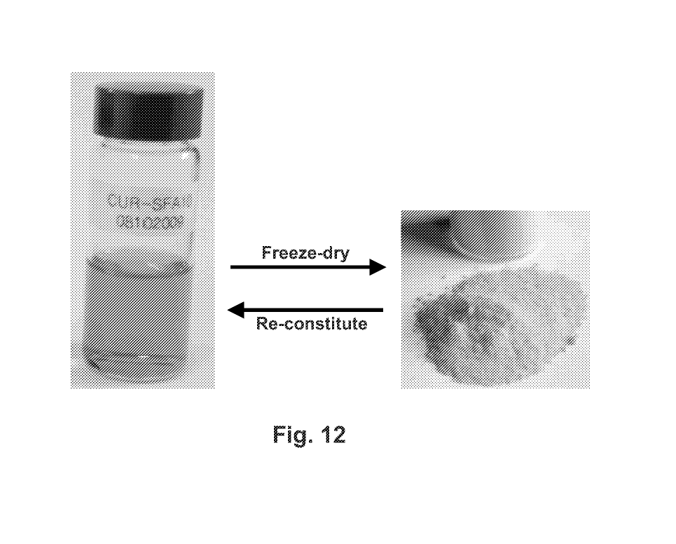

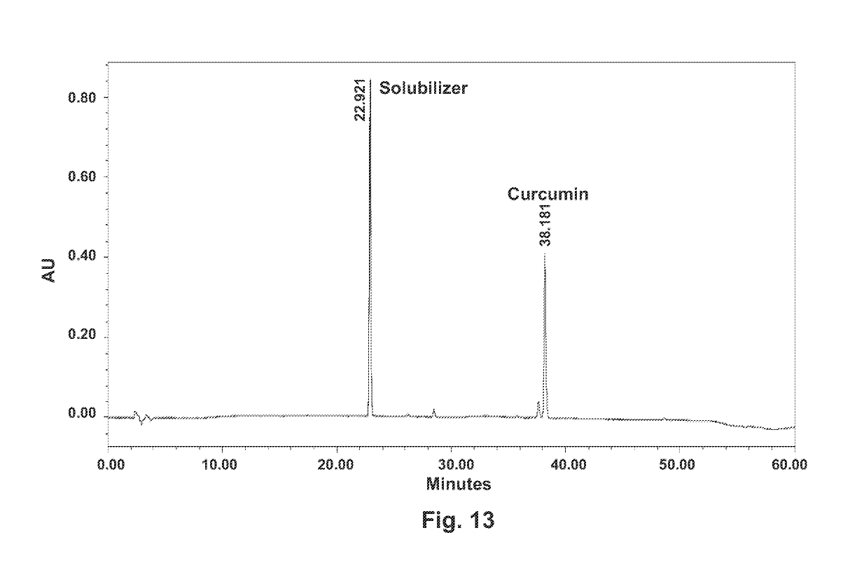

The 30% and 40% curcumin-rubusoside water solution (10 mL) were freeze-dried to powder. The powders appeared golden-colored. FIG. 12 shows the powder and the initial 10% curcumin-rubusoside solution, both which were a deep yellow color. The 40% curcumin-rubusoside water solution (10 mL) was freeze-dried to powder (4.2 gm). Part of this powder (100 mg) was re-constituted in 10 mL water. The powder completely dissolved. FIG. 13 shows the HPLC chromatogram of the reconstituted curcumin-solubilizer water solution. The solubilizer and curucmin were eluted at 22.9 min and 38.1 min, respectively. Curcumin used had purity (HPLC) of 95%, and the solubilizer of 98%. This HPLC analysis showed the reconstituted solution had a curcumin content of 2.03% w/w or a concentration of 203.3 μg/mL, and 11.46 mg/mL rubusoside. (FIG. 13) Similarly, the 30% curcumin-rebusoside solution was freeze-dried to powder, and 100 mg powder reconstituted with 10 mL water. The curcumin content of this solution was measured as 1.87% w/w.

Reconstituted Curcumin Retained Cytotoxic Activity.

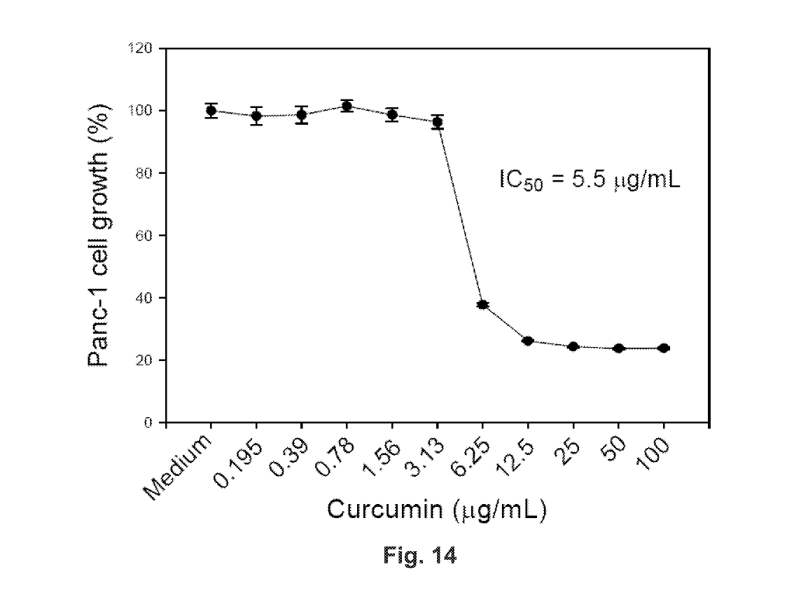

The re-constituted curcumin water solution was adjusted to pH 7.2 with 9.55 mg/ml PBS powder to make the final concentration about 100% or 1×. The pH-adjusted water solution was then co-cultured with human pancreatic cancer cells (PANC-1) for 72 hours in a standard cell culture medium. PANC-1 was obtained from the American Type Culture Collection (ATCC) and was maintained at 37° C. in a humidified atmosphere with 5% CO₂. PANC-1 cells were cultured in Dulbecco's Modified Eagle's Medium (DMEM) supplemented with 10% fetal bovine serum, HEPES, penicillin-streptomycin, sodium pyruvate, L-glutamine, and non-essential amino acids. All culture materials were purchased from Invitrogen Corporation (Carlsbad, Calif.). Curcumin at a concentration below 3.13 μg/mL had no inhibitory effect. However, 6.25 μg/mL curcumin showed strong inhibition. (FIG. 14). Maximum growth inhibition was achieved at curcumin concentrations of 12.5 μg/mL and higher. The IC50 was calculated to be about 5.5 μg/mL (14.9 μM) curcumin which is in the same range of 17 μM for Panc-1 reported by Holcomb et al. in J Gastrointest Surg (2008) 12:288-296. Thus the freeze-drying and reconstituting of the curcumin-rubusoside solution did not affect the effectiveness of the curcumin.

### Example 7

Formation of Paclitaxel-Solubilizer Complexes in Dried Powder and its Re-Constitution to Water Solutions.

A 10% w/v rubusoside water solution was prepared. Five milligrams of paclitaxel was weighed into a flask, and 10 mL of the 10% rubusoside solution was added. The solution was vortexed briefly and then sonicated for 60 min at temperature of 50° C., and then subjected to a heat and pressure treatment composed of 121° C. and 1.1 to 1.2 atmospheric pressure for 60 min. The solution was placed in an incubator set at 25° C. for at least 24 hr, and then centrifuged at 4000 rpm for 10 min. The supernatant solution was passed through a 0.45 μm filter and analyzed for the concentration of paclitaxel by HPLC or LC/MS. Quantification was done by comparing to a standard solution of a known amount in methanol. In the presence of 10% w/v rubusoside, the solution contained 269 μg paclitaxel/mL water.

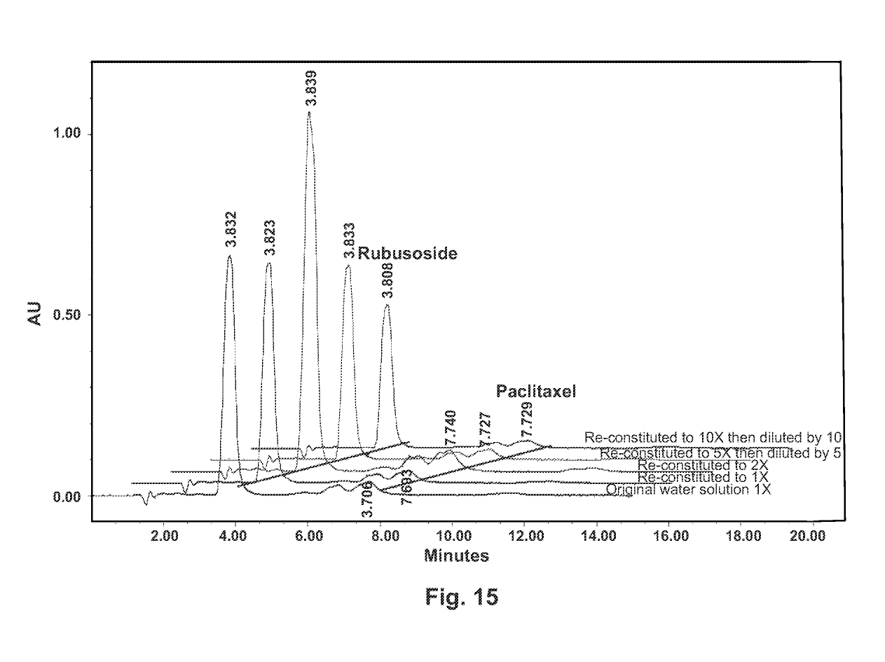

Four aliquots of the 10% paclitaxel-rubusoside water solution (1 mL each) was freeze-dried to powder in four separate 1 mL tubes. The powder appeared as white crystals. The powder was reconstituted using 1 ml, 0.5 ml, 0.25 ml or 0.1 ml water, followed by 60 min sonication and a brief vortex. In all cases, the powder completely dissolved. FIG. 15 shows the chromatograms of reconstituted paclitaxel water solutions containing rubusoside as a solubilizing agent. The HPLC-MS (Waters HPLC-MS system with 600 pump, 717 autosampler, 2996 PDA, and an EMD1000 MS detector) chromatograms were generated using a Prevail C18 column (2.1×150 mm, 3 μm) and a mobile phase of 0.25% HCOOH:ACN:MeOH (4:4:2 v/v/v); at a flow rate of 0.40 mL/min, injection volume of 1 μL, UV detection wavelength of 230 nm, and column temperature of 30 C. MS detection was performed with MS-ESI in positive mode; a full scan range of m/z 200-960 and a SIR scan at m/z 854.4. Paclitaxel concentrations were determined using a standard paclitaxel calibration curve with paclitaxel standard in methanol. The chromatograms were generated at combined 230 nm and 215 nm UV showing elution of rubusoside at 3.8 min and paclitaxel at 7.7 min. The resulting concentrations of paclitaxel in the reconstituted solutions at 1 ml, 0.5 ml, 0.25 ml and 0.1 ml water were 187 μg/mL, 318 μg/mL, 770 μg/mL, and 1102 μg/mL, respectively. (FIG. 15). Thus the paxlitaxel could be concentrated by several fold depending on the amount of water used to reconstitute. The discrepancy in the paclitaxel concentrations between the original solution and the reconstituted solution are believed to be sampling errors associated with small samples.

### Example 8

Solubility and Formation of Fat-Soluble Compounds-Solubilizer Complexes in Dried Powder and its Re-Constitution to Water Solutions.

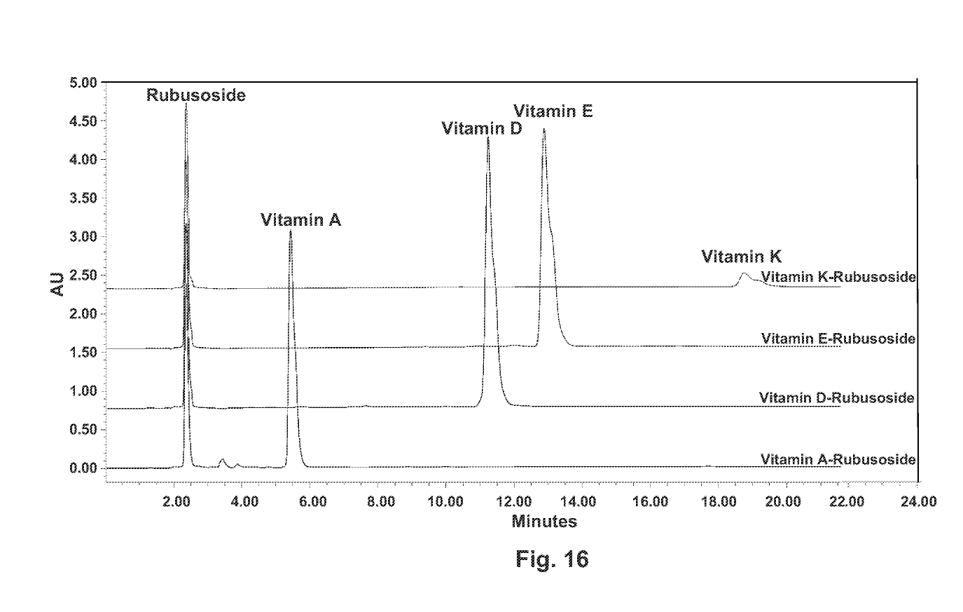

A 10% w/v rubusoside water solution was prepared. Various amounts of fat-soluble vitamins (e.g., vitamin A, vitamin D₃ (cholecalciferol), vitamin E (alpha-tocopherol), and vitamin K₁ (phylloquinone)) were each weighed into separate flasks, and 10 mL rubusoside solution was added to each. The prepared water solutions were vortexed briefly and then sonicated for 60 min at temperature of 50° C. The water solutions of vitamins D3, E, and K1 were further subjected to a heat and pressure treatment composed of 121° C. and 1.1 to 1.2 atm for 60 min, then placed in an incubator at 25° C. for 24 hours. The solution of vitamin A was placed directly in a rotating shaker in an incubator set at 25° C. for 48 hours. All solutions were then centrifuged at 4000 rpm for 10 min. The supernatant of each solution was passed through a 0.45 μm filter and analyzed for concentration by HPLC or LC/MS. The color of the filtered solutions was clear. Quantification was done by comparing to a standard solution of a known amount in methanol. FIG. 16 shows chromatograms of fat-soluble vitamins A, D, E, and K in water solutions containing 10% w/v rubusoside as complexing agent. The HPLC (Waters HPLC system with 1525 pump, 717 autosampler, and 2996 PDA) chromatograms were generated using a phenomenex Luna C18 column (4.6×250 mm, 5 μm) and a mobile phase of CH3OH:H2O, the gradient was CH3OH from 93% to 100% in 7 min at a flow rate of 1.5 mL/min, injection volume of 5 μL, PDA detection wavelength of 200-600 nm, and column temperature of 30 C. Vitamin concentrations were determined using respective standard vitamin methanol solutions of 1 mg/mL. The chromatograms of Vitamin A-Rubusoside, Vitamin D-Rubusoside, Vitamin E-Rubusoside, and Vitamin K-Rubusoside were generated at combined 215 nm with 327, 265, 293 or 270 nm UV for each corresponding vitamin. Rubusoside was eluted at 2.3 min, vitamin A at 5.4 min, vitamin D at 11.3 min, vitamin E at 12.9 min and vitamin K at 18.8 min. Vitamin concentrations in the solubilized water solutions were: 1.3 mg/mL vitamin A, 4.5 mg/mL vitamin D3, 9.9 mg/mL vitamin E, and 791 μg/mL vitamin K₁ (FIG. 16).

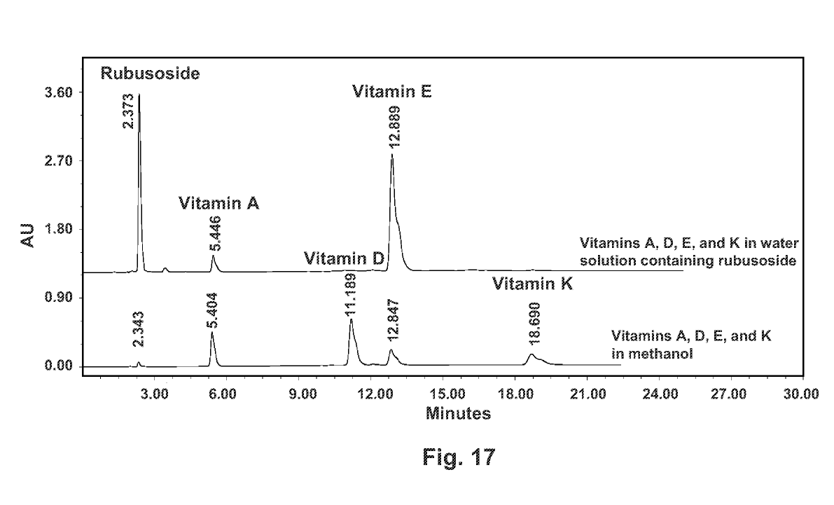

The recommended daily dietary allowances for males of age 19-70 (Dietary Reference Intakes: Vitamins. The National Academies, 2001) for these four fat-soluble vitamins are 900 μg vitamin A, 5.0-10.0 μg vitamin D, 15.0 mg Vitamin E, and 120 μg Vitamin K. A single solution was prepared to achieve these dietary allowance concentrations using the above solutions of the vitamins with 10% rubusoside: 2.7 mL vitamin A solution, 0.3 mL vitamin D solution, 4.5 mL vitamin E solution, and 0.4 mL vitamin K solution, totaling a volume of 7.9. FIG. 17 shows chromatograms of this mixed vitamin water solution containing fat-soluble vitamins A, D, E, and K in the presence of 10% w/v rubusoside at the combined wavelengths of 215 nm and 270 nm and a mixed methanol solution containing 250 μg/mL each of vitamins A, D, E, and K. The HPLC (Waters HPLC system with 1525 pump, 717 autosampler, and 2996 PDA) Chromatograms were generated using a phenomenex Luna C18 column (4.6×250 mm, 5 μm) and a mobile phase of CH3OH:H2O, the gradient was CH3OH from 93% to 100% in 7 min at a flow rate of 1.5 mL/min, injection volume of 5 μL, PDA detection wavelength of 200-600 nm, and column temperature of 30 C. Each vitamin concentration was determined using a standard vitamin in methanol at 1 mg/mL. Elution of rubusoside occurred at 2.3 min; vitamin A at 5.4 min, vitamin D at 11.2 min, vitamin E at 12.3 min, and vitamin K at 18.7 min. HPLC analysis found that the re-composed single water solution contained 392.0 μg/mL vitamin A, 4.7 μg/mL vitamin D, 5.839 mg/mL vitamin E, 34.4 μg/mL vitamin K, and 100 mg/mL rubusoside (FIG. 17). To reach the range of daily allowance concentrations, 2.3 mL of the recomposed solution would be needed to deliver 902 μg vitamin A, 10.8 μg vitamin D, 13.43 mg Vitamin E, and 79 μg Vitamin K, along with 230 mg rubusoside.

In FIG. 17, the lower chromatogram shows the retention of each vitamin each at a concentration of 250 μg/mL in methanol, and the top chromatogram shows the recomposed solution containing vitamins A, D, E, and K and rubusoside.

The water solution of the re-composed mixture of the four fat-soluble vitamins and rubusoside was freeze-dried to powder, and then completely re-constituted in water. It is believed this method of solubilizing would be effective for other fat-soluble compounds or mixtures of fat-soluble compounds.

### Solubilizing Agent as P-gp Inhibitor

### Example 9

Rubusoside as a P-gp Inhibitor

A natural solubilizing agent, rubusoside, was found to inhibit permeability glycoprotein (P-gp or Pgp). At 1 mg/mL (1.558 mM) concentration, rubusoside (SFA) showed a 59% inhibition to P-pg in a Caco-2 assay, a pharmaceutical industry gold standard for predicting human gastrointestinal absorption.

Rubusoside:

Rubusoside was extracted from Chinese sweet leaf tea leaves (Rubus suavissimus; Rosaceae) purchased from Natural Plants Products Factory, Guilin S&T New Tech Company, Sanlidian Campus of Guangxi Normal University, Guilin, Guangxi, China. This extraction process is described in PCT/US2009/040324, International Published Application WO 2009/126950. Rubusoside has a molecular formula C32H50O13 and molecular weight of 642.73. First, the air-dried leaves were boiled with water with a weight to volume ratio ranging from about 1:10 to about 1:20. From this extraction, a crude dried extract (20 to 30% dry weight yield from the raw leaves) was obtained that contained from about 5% to about 15% rubusoside by weight. The dried extract was then reconstituted with water to a weight to volume ratio ranging from about 1:4 to about 1:5. In this concentrated extract, the ellagitannins would partially precipitate out and were removed by filtration. The rubusoside was retained in the solution. The solution containing rubusoside was then subjected to column chromatography using a macroporous resin (Dowex Optipore L493 Polymeric Adsorbent, Styrene-Divinylbenzene polymers with 46 Angstrom average pore size; The Dow Chemical Company, Midland, Mich.). The column was eluted with ethanol to obtain a purified extract containing approximately 60% rubusoside and about 1% steviol monoside. Subsequently, the purified extract was loaded on a second column to further purify the extract using silica gel as the stationary absorbent (Silica Gel, 200-300 mesh, Natland International Corporation, Research Triangle, N.C.). The column was eluted with a mixed solvent (chloroform:methanol at a ratio of 8:2 v/v). The extract from this second column was at least 80% pure rubusoside, and was dried to a powder. Finally, this rubusoside-rich extract (>80% w/w) was dissolved in absolute methanol by heating to temperatures ranging from about 60° C. to about 80° C. The solution was then cooled to allow re-crystallization of rubusoside. This re-crystallization process may need to be repeated to obtain pure rubusoside (>99% purity as measured on HPLC). The structure of rubusoside was confirmed by mass spectrometry and NMR. Rubusoside, a diterpene glycoside, has a molecular weight of 642 Daltons, and is a white crystal or powder. The crystalline powder is stable at temperatures ranging from about −80° C. to over 100° C. In water, rubusoside itself has a solubility of approximately 400 mg/ml at 25° C. and 800 mg/ml at 37° C., which is greater than that of many common, water-soluble compounds (e.g., sodium chloride has a solubility of 360 mg/ml water).

Powdered rubusoside was sent to a commercial contract laboratory, Apredica, Watertown, Mass., for testing for P-gp inhibition. Test agent powders were stored at −20° C. Samples were analyzed by LC/MS/MS using either an Agilent 6410 mass spectrometer coupled with an Agilent 1200 HPLC and a CTC PAL chilled autosampler, all controlled by MassHunter software (Agilent), or an ABI2000 mass spectrometer coupled with an Agilent 1100 HPLC and a CTC PAL chilled autosampler, all controlled by Analyst software (ABI). After separation on a C18 reverse phase HPLC column (Agilent, Waters, or equivalent) using an acetonitrile-water gradient system, peaks were analyzed by mass spectrometry (MS) using ESI ionization in MRM mode.

Human epithelial colorectal adenocarcinoma cell line (Caco-2) was obtained from the American Type Culture Collection (ATCC), and cultured according to directions. Caco-2 cells grown in tissue culture flasks were trypsinized, suspended in medium, and the suspensions were applied to wells of a collagen-coated BioCoat Cell Environment in 24-well format (BD Biosciences) at 24,500 cells per well. The cells were allowed to grow and differentiate for three weeks, feeding at 2-day intervals. To measure inhibition of P-gp transporter activity, permeability and efflux ration of a probe P-gp substrate (5 μM digoxin) were determined after preincubation of the cells with the test agent or vehicle. For Apical to Basolateral (A->B) permeability, the test agent (1 mg/mL)+probe substrate were added to the apical (A) side, test agent was added to the Basolateral (B) side, and amount of permeation was determined on the B side. For Basolateral to Apical (B>A) permeability, the test agent+probe substrate were added to the B side, test agent was added to the A side, and the amount of permeation of probe substrate was determined on the A side. Buffer alone, without test agent, was used as a negative control. Verapamil was used as a positive inhibitor control. The A-side buffer contained 100 μM Lucifer yellow dye, in Transport Buffer (1.98 g/L glucose in 10 mM HEPES, 1× Hank's Balanced Salt Solution) pH 6.5, and the B-side buffer was Transport Buffer, pH 7.4. Dosing solutions were prepared by dissolving the test agent in Transport buffer, then adding Lucifer yellow to a portion as indicated. CaCo-2 cells were incubated with these buffers for 2 h., and the receiver side buffer is removed for analysis by LC/MS/MS.

To verify the CaCo-2 cell monolayers were properly formed, aliquots of the cell buffers were analyzed by fluorescence to determine the transport of the impermeable dye Lucifer Yellow. A Lucifer yellow rejection ratio>99% is expected. Exceptions are noted in the results.

$$Data a r e expressed as permeability ( {P}_{app} ) \text{:} {P}_{app} = \frac{d Q / d t}{{C}_{0} A}$$

Where dQ/dt is the rate of permeation, C₀ is the initial concentration of test agent, and A is the area of the monolayer.

From bidirectional permeability studies, the efflux ration (RE) is also calculated:

$${R}_{E} = \begin{matrix}{P}_{app} \\ {P}_{app}\end{matrix} \frac{( B \to A ) .}{( A \to B ) .}$$

A reduction in R_{E} in the presence of test compounds indicates P-gp inhibition.
The results are shown in the following tables.

TABLE 4
P-gp Inhibition Summary

| | | | Assay | Ef- | | |
|---|---|---|---|---|---|---|
| Test | Test | | duration | flux | Inhibi- | |
| Inhibitor | Conc | | (hr) | ratio^{a} | tion | Comment |
| Rubusoside | 1 | mg/mL | 2 | 6.3 | 59% | |
| Vehicle | | | 2 | 13.7 | 0% | Negative |
| | | | | | | control (0% |
| | | | | | | inhibition) |
| Verapamil | 100 | μM | 2 | 1.2 | 100% | Positive |
| | | | | | | control (100% |
| | | | | | | inhibition) |

^{a}Pₐₚₚ (B −> A)/Pₐₚₚ (A −> B)

TABLE 5
Caco-2 permeability

| | Probe | | | | | |
|---|---|---|---|---|---|---|
| Probe | substrate | Assay | | | | |
| substrate/test | conc | duration | Pₐₚₚ. A-B | Pₐₚₚ. B-A | Efflux | |
| inhibitor | (μM) | (hr) | (10⁻⁶ cm s⁻¹) | (10⁻⁵ cm s⁻¹) | ratio | Comment |
| Digoxin/ | 5 | 2 | 2.3 | 14.1 | 6.3 | |
| 1 mg/mL | | | | | | |
| Rubusoside | | | | | | |
| Digoxin/ | 5 | 2 | 1.4 | 18.7 | 13.7 | Negative control |
| Vehicle | | | | | | (0% inhibition) |
| Digoxin/ | 5 | 2 | 5.3 | 6.2 | 1.2 | Positive control |
| 100 μM | | | | | | (100% inhibition) |
| verapamil | | | | | | |
| Ranitidine/ | 10 | 2 | 1.7 | | | Low permeability |
| Vehicle | | | | | | control |
| Warfarin/ | 10 | 2 | 33.5 | | | High permeability |
| vehicle | | | | | | control |

The P-gp inhibition data were calculated from the permeability results for the probe substrate as indicated. Ranitidine (low permeability) and warfarin (high permeability) are included as controls to ensure the proper performance of the test system.

Rubusoside was shown to inhibit P-gp, and would thus improve intestinal absorption of several drugs across the gastrointestinal epithelium and into the blood stream.

The term “effective amount” as used herein refers to an amount of rubusoside sufficient to inhibit permeability glycoprotein and increase intestinal absorption of a compound to a statistically significant degree (p<0.05).

The complete disclosures of all references cited in this specification are hereby incorporated by reference, including U.S. provisional patent application Ser. Nos. 61/251,768 and 61/314,800. In particular, the disclosure of International Patent Application No. PCT/US2009/040324, published as International Published Application No. WO 2009/126950, is completely incorporated into this application. In the event of an otherwise irreconcilable conflict, however, the present specification shall control.

1. A process to increase the solubility of one or more organic compounds which are insoluble or sparingly soluble in water, said process comprising homogenizing said compounds with water and a diterpene glycoside and subjecting the homogenized mixture to increased temperature and pressure.

2. The process of claim 1, wherein the diterpene glycoside is selected from the group consisting of steviol glycoside, rubusoside, stevioside, and rebaudioside A.

3. The process of claim 1, wherein the diterpene glycoside is rubusoside.

4. The process of claim 1, wherein the diterpene glycoside is rebaudioside A.

5. The process of claim 1, wherein the temperature is from about 100° C. to about 200° C. and the pressure from about 1.1 to about 3.2 atm.

6. The process of claim 1, wherein the homogenization is at a speed from about 4000 rpm to about 22,000 rpm for a time from about 30 seconds to about 20 minutes.

7. The process of claim 1, wherein the one or more insoluble or sparingly soluble organic compounds are selected from the group consisting of diterpenes, quinoline alkaloids, fat-soluble compounds, and curcuminoids.

8. The process of claim 1, wherein the insoluble or sparingly soluble organic is paclitaxel.

9. The process of claim 1, wherein the insoluble or sparingly soluble organic is camptothecin.

10. The process of claim 1, wherein the insoluble or sparingly soluble organic is curcumin.

11. The process of claim 1, wherein the insoluble or sparingly soluble organic is rutin.

12. A method for solubilizing one or more fat-soluble compounds in water using a diterpene glycoside to increase the solubility of the compounds in water by a factor of 2 or more.

13. The method of claim 12, wherein the fat-soluble compound is a fat-soluble vitamin.

14. The method of claim 13, wherein the fat-soluble vitamin is selected from the group consisting of vitamin A, vitamin D3 (cholecalciferol), vitamin E (alpha-tocopherol), and vitamin K1 (phylloquinone).

15. The method of claim 12, wherein the diterpene glycoside is rubusoside.

16. A dry composition comprising one or more complexes of a diterpene glycoside and one or more organic compounds which are insoluble or sparingly soluble in water; wherein the powder completely dissolves when mixed with an aqueous solution.

17. The composition of claim 16, wherein said diterpene glycoside is selected from the group consisting of rubuososide, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

18. The composition of claim 16, wherein the diterpene glycoside is rubusoside.

19. The composition of claim 16, wherein the diterpene glycoside is rebaudioside A.

20. The composition of claim 16, wherein the organic compound is selected from the group consisting of diterpenes, quinoline alkaloids, phenylalanine-derived alkaloids, hydrolysable tannins, flavonoids, curcuminoids, phenols, quinones, macrolides, cyclic peptides, sesquiterpene lactones, lignans, flavonolignans, lipids, fat-soluble compounds, and azoles.

21. The composition of claim 16, wherein the organic compound is selected from the group consisting of curcumin, paclitaxel, rutin, progesterone, resveratrol, vitamin A, vitamin D, vitamin E, and vitamin K.

22. The composition of claim 16, wherein the composition has two or more complexes wherein the organic compound is selected from the group consisting of vitamin A, vitamin D, vitamin E, and vitamin K.

23. A composition comprising an aqueous solution of one or more fat-soluble compounds and a diterpene glycoside; wherein the concentration of said diterpene glycoside is sufficient to increase the solubility of said compound in water by a factor of 2 or more above what the solubility of said compound would be in an otherwise identical composition lacking said diterpene glycoside.

24. The composition of claim 23, wherein said diterpene glycoside is selected from the group consisting of rubuososide, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

25. The composition of claim 23, wherein the diterpene glycoside is rubusoside.

26. The composition of claim 23, wherein the fat-soluble compound is a fat soluble vitamin.

27. The composition of claim 26, wherein the fat-soluble vitamin is selected from the group consisting of vitamin A, vitamin D, vitamin E, and vitamin K.

28. The composition of claim 23, wherein the composition has two or more complexes wherein the vitamin is selected from the group consisting of vitamin A, vitamin D, vitamin E, and vitamin K.

29. A method to increase the intestinal absorption of a compound, said method comprising orally administering concurrently with the compound an effective amount of rubusoside.

30. The method as in claim 29, wherein said compound is a drug.

31. The method as in claim 29, wherein the drug is selected from the group consisting of digoxin, paclitaxel, and etoposide.

32. A method to inhibit the activity of permeability glycoprotein (P-gp) in the intestine of a subject, said method comprising orally administering to the subject an effective amount of rubusoside.

33. A method to inhibit the activity of permeability glycoprotein (P-gp) in cells expressing P-gp, said method comprising administering to the cells an effective amount of rubusoside.

34. The method of claim 33, wherein the cells expressing P-gp are selected from the group consisting of intestinal epithelium cells, renal proximal tubular cells, adrenal gland cells, and capillary endothelial cells.
